(19) United States
(12) Patent Application Publication
(10) Pub. No.: US 2010/0031452 A1
(43) Pub. Date: Feb. 11, 2010

(54) AZO DYES

(75) Inventors: Estelle Enaud, Mont-Saint-Guibert (BE); Christian-Marie Bols, Chastre (BE); Ana Casas Infantes, Fraga (Huesca) (ES); Sylvie Groslambert, Liege (BE); Christoph Hercher, Müllheim (DE); Gilles Iacazio, Marseille (FR); Jutta Ismene Jager, Umkirch (DE); Jacqueline Marchand-Brynaert, Marcinelle (BE); Rezzan Karaaslan, Istanbul (TR); Mehmet Emre Sener, Istanbul (TR); Thierry Tron, Marseille (FR); Sophie Vanhulle, Chastre (BE)

Correspondence Address: KNOBBE MARTENS OLSON & BEAR LLP, 2040 MAIN STREET, FOURTEENTH FLOOR, IRVINE, CA, 92614, US

(73) Assignees: Universite Catholique de Louvain, Louvain-la-Neuve (BE); Wetlands Incubator sprl, Louvain-la-Neuve (BE)

(21) Appl. No.: 12/498,999

(22) Filed: Jul. 7, 2009

Related U.S. Application Data
(60) Provisional application No. 61/078,675, filed on Jul. 7, 2008.

(51) Int. Cl. A61K 8/41; C09B 56/12; C12P 15/00; D06P 3/32; A61Q 5/10

(52) U.S. Cl. 8/405; 534/656; 534/654; 435/127; 8/437

(57) ABSTRACT

The present invention relates to azo dyes, in particular azo dyes of Formula (I) or (II), a tautomer, a quaternary amine, or a salt thereof,

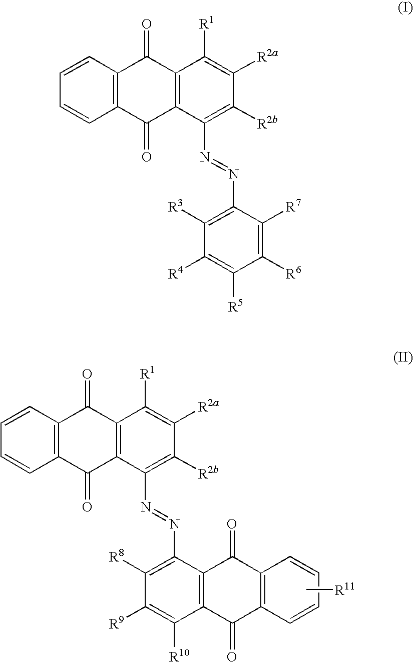

wherein R¹, R^{2a}, R^{2b}, R³, R⁴, R⁵, R³, R⁷, R⁸, R⁹, R¹⁰, R¹¹ have the same meaning as in the claims. The present invention also relates to processes for preparing said compounds, compositions containing them and their use as dyes.

### RELATED APPLICATIONS

This application claims priority to U.S. Provisional Application No. 61/078,675, filed Jul. 7, 2008, which is incorporated herein by reference in its entirety.

### FIELD OF THE INVENTION

The present invention relates to new azo dyes, preparation methods for these azo dyes and formulations for dyeing fibers, such as, keratine fibers for example, human hair, wool or furs, comprising these compounds.

### BACKGROUND OF THE INVENTION

The European Commission started on July 2007 a new EU regulatory framework for chemicals. Under this new system called REACH (Registration, Evaluation, Authorization and Restriction of Chemicals), enterprises that manufacture or import more than one ton of a chemical substance per year would be required to register it in a central database. This enhances the constraints for the color industry. Many dyes have demonstrated toxicity in the past. The new dyes developed have to prove their safety through adequate toxicity studies. Indeed, several parameters have to be tested as dyes present on the market (not cytotoxic) were recently proved to be mutagenic (Jager et al Mutation Research/Genetic Toxicology and Environmental Mutagenesis 2004. 561(1-2): 35-44; Jager et al. Melliand English 2005: E12-E14; Schneider et al. Journal of Applied Toxicology 2004. 24: 83-91).

Azo dyes constitute the largest group of dyes, both with reference to the number of different chemical structures and to the total production volume (Zollinger H, Color Chemistry: syntheses, properties and applications of organic dyes and pigments. 2003, Weinheim: VCH). They are extensively used in textile dyeing and paper printing because of their great color variety. They generally present high molar extinction coefficients and medium to high light and wet fastness properties (Zollinger 2003). A survey of oral acute toxicity of 4461 dyes as measured by the 50% lethal dose has revealed that azo and cationic dyes are the most toxic, and there is ample evidence of the mutagenicity of certain dyes, especially azo dyes.

Developing new less toxic and non mutagenic azo dyes is therefore needed.

Additional problems are that the chemical synthesis pathways of traditional azo dyes are usually non worker-friendly processes and non environmental friendly processes.

Azo dyes are generally synthesized through diazotization followed by azo coupling. In general, diazotization of an aromatic or heteroaromatic primary amine is usually carried out at 0° C., due to the explosive character of the compounds, in presence of sodium nitrite and with an excess of mineral acid such as HCl, H₂SO₄, or HBF₄. In the second step, the coupling to basic components requires higher pH. Such syntheses pose an environmental problem as high amounts of electrolytes are produced for the neutralization of the reaction mixture and purification of the dye, and sometimes, low yields result in highly colored effluents. Furthermore, the use of strong mineral acids is non worker friendly and the cooling under 5° C. required during the first step results in high energy consumption.

U.S. Pat. No. 4,709,019 describes anthraquinone azo pyridine containing compounds, and the synthesis thereof. The reactions are performed in nitrosylsulfuric acid or sulfuric acid in a sodium nitrite solution. These reactions are not worker friendly conditions and include steps at 0° C. and steps between 70° C. and 90° C., which is energivoracious and not environmental friendly.

GB 330644 describes the synthesis of azoanthraquinone. The azoanthraquinone is formed from diaminoanthraquinone derivatives by oxidation using chromic acid, which is a non environmental friendly process. Chromium (IV) compound are carcinogenic and toxic. For this reason chromic acid oxidation is not used at the industrial scale.

There is therefore a need to find improved methods for the preparation of azo dye, which are worker and environmentally friendly. It is accordingly one of the objects of the present invention to overcome or ameliorate at least one of the disadvantages of the prior art, or to provide a useful alternative.

### SUMMARY OF THE INVENTION

An objective of the present invention is therefore to provide new azo dyes, wherein at least one of the above mentioned drawbacks is overcome. In particular, it is an object of the present invention to provide new azo dyes amongst which some are less toxic than prior art dyes. It is a further object of the invention to provide method for the preparation of azo dyes which solves at least one of the above mentioned drawbacks.

The present invention concerns a compound having the structural Formula (I) or (II), a tautomer, a quaternary form, or a salt thereof,

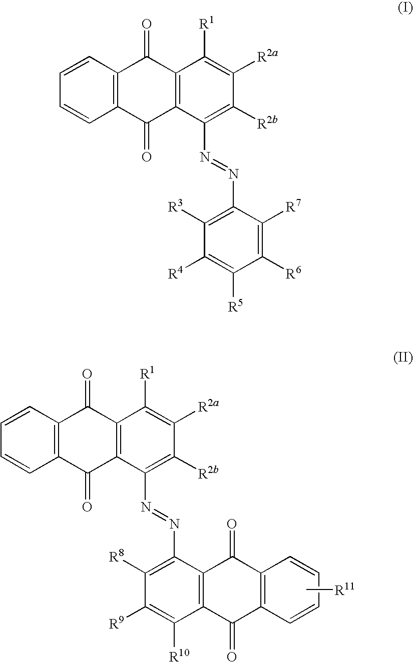

wherein

R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂;

R^{2a} is hydrogen or —SO₃H;

R^{2b} is hydrogen or —SO₃H;

wherein at least one of R^{2a} or R^{2b} is —SO₃H;

R³ is selected from the group consisting of hydrogen, halogen, —SO₃H, C₁₋₆alkyl, amino, C₁₋₆alkylamino, C₆₋₁₂arylamino, C₁₋₆alkoxy, aryloxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂;

R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, and C₁₋₆alkylaminocarbonyl;

R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁶ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, —C(OH)═CH—OH, and —NH-phenylene-SO₂—CH═CH₂;

R⁸ is hydrogen or —SO₃H;

R⁹ is hydrogen or —SO₃H;

R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and

R¹¹ is selected from the group consisting of hydrogen, C₁₋₆alkyl, amino, halogen, hydroxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl.

In an particular embodiment, the present invention concerns a compound having the structural Formula (I) or (II), a tautomer, a quaternary form, or a salt thereof,

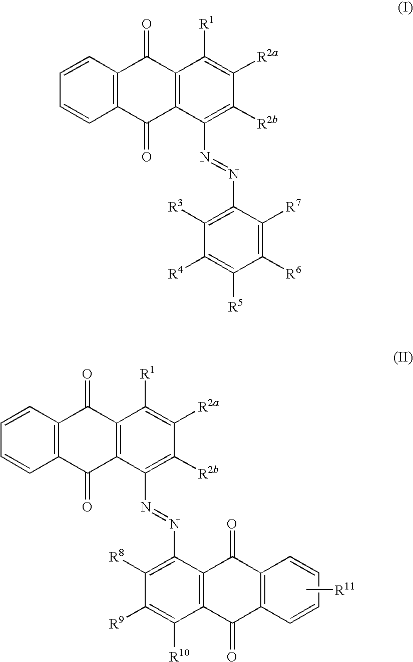

wherein

R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂;

R^{2a} is hydrogen or —SO₃H;

R^{2b} is hydrogen or —SO₃H;

wherein at least one of R^{2a} or R^{2b} is —SO₃H;

R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂;

R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁶ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, —C(OH)═CH—OH, and —NH-phenylene-SO₂—CH═CH₂;

R⁸ is hydrogen or —SO₃H;

R⁹ is hydrogen or —SO₃H;

R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and

R¹¹ is selected from the group consisting of hydrogen, C₁₋₆alkyl, amino, halogen, hydroxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl.

Some of the compounds according to the present invention have the advantage of being non mutagenic, and present a low cytoxicity and low ecotoxicity in the range of conditions tested. Some of the compounds have also a good optical density at the maximum wavelength.

Their method of production is preferably based on coupling by an oxidoreductase of precursors with aromatic amines structures in mild conditions. They provide the advantages of being safer and environmentally friend routes to azo dyes.

In particular, the present invention also concerns a process for the production of a compound of the invention, comprising the step of: coupling an amine of formula (Ia) with a compound of formula (Ib) or (IIb) in the presence of an oxidoreductase;

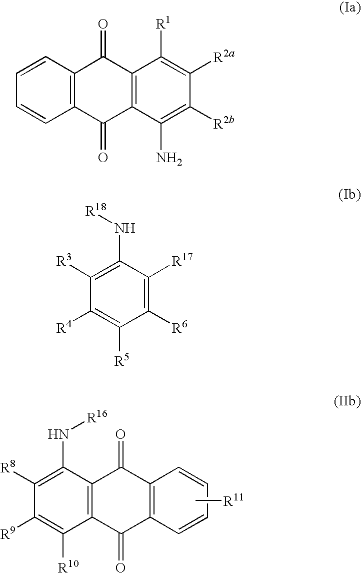

wherein R¹⁶ is selected from the group comprising hydrogen, C₁₋₆cycloalkyl, C₆₋₁₀aryl, haloC₆₋₁₀aryl, 9,10-dioxoanthracenyl, or 4-p-tolyloxysulfonyloxy-phenyl;

R¹⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, carboxyl and —NH-phenylene-SO₂—CH═CH₂; and

R¹⁸ is selected from hydrogen, or C₆₋₁₂aryl; or

NH—R¹⁸ and R¹⁷ together with the carbon atom to which they are attached form a 5 membered heteroaryl or heterocyclyl ring, each ring being optionally substituted with one or two substituents selected from C₁₋₆alkylcarbonyloxy or carboxyl;

wherein R¹, R^{2a}, R^{2b}, R³, R⁴, R⁵, R⁶, R⁸, R⁹, R¹⁰, and R¹¹ have the same meaning as defined herein.

The present invention provides a process that can be applied at reduced reaction temperature comparing to prior art conditions. In an embodiment, the pH of the present process is also milder than traditional chemical synthesis. The present process has the advantages of avoiding the use of dangerous reactant or the production of dangerous or explosive component and therefore provides advantage towards the reduction of the ecological footprint in industrial processes.

The compounds of the present invention can be used as dyes for textile, leather, hair, cosmetic or paper applications, or for biological staining. Beside the synthesis of new compounds, these findings open the way towards safe and environmentally friend routes to azo dyes.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### BRIEF DESCRIPTION OF THE DRAWINGS

FIGS. 1 and 2 represent graphs plotting the optical density measured for some compounds according to the invention.

FIG. 3 represent a graph plotting the results of toxicity measured for some compounds according to the invention.

FIG. 4 represent pictures of fabric samples dyed with compounds according to embodiment of the invention.

### DETAILED DESCRIPTION

The present invention concerns azo compounds of Formula (I) or (II), a tautomer, a quaternary amine, or a salt thereof, wherein

R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂; preferably, R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, and —N═N—C₆₋₁₂arylene-SO₃H; preferably R¹ is selected from the group consisting of hydrogen, C₁₋₆alkyl, C₃₋₈cycloalkylamino, amino, hydroxyl, C₁₋₆alkylamino, C₆₋₁₀arylamino, haloC₆₋₁₀arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₀arylene-hydroxyl, —N═N—C₆₋₁₀arylene-SO₃H, and —SO₂—CH═CH₂; preferably R¹ is selected from the group consisting of hydrogen, C₁₋₆alkyl, C₃₋₈cycloalkylamino, amino, hydroxyl, C₁₋₆alkylamino, C₆₋₁₀arylamino, haloC₆₋₁₀arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₀arylene-hydroxyl, and —N═N—C₆₋₁₀arylene-SO₃H; preferably, R¹ is selected from the group consisting of hydrogen, C₁₋₄alkyl, C₄₋₆cycloalkylamino, amino, hydroxyl, C₁₋₄alkylamino, C₆₋₁₀arylamino, haloC₆₋₁₀arylamino, C₁₋₄alkoxy, C₁₋₄alkoxyC₁₋₆alkyl, C₁₋₄alkoxycarbonyl, aminocarbonyl, C₁₋₄alkylaminocarbonyl, —N═N—CH═CH—C₆₋₁₂arylene-hydroxyl, —N═N—C₆₋₁₀arylene-SO₃H, and —SO₂—CH═CH₂; preferably, R¹ is selected from the group consisting of hydrogen, C₁₋₆alkyl, C₃₋₈cycloalkylamino, amino, C₁₋₆alkylamino, C₆₋₁₂arylamino, haloC₆₋₁₂arylamino, C₁₋₆alkoxy, C₁₋₆alkoxycarbonyl, aminocarbonyl, C₁₋₆alkylaminocarbonyl, —N═N—CH═CH—C₆₋₁₂arylene-hydroxyl, and —N═N—C₆₋₁₂arylene-SO₃H; more preferably, R¹ is selected from the group consisting of hydrogen, C₃₋₈cycloalkylamino, amino, C₁₋₆alkylamino, C₆₋₁₂arylamino, haloC₆₋₁₂arylamino, aminocarbonyl, C₁₋₆alkylaminocarbonyl, —N═N—CH═CH—C₆₋₁₀arylene-hydroxyl, and —N═N—C₆₋₁₀arylene-SO₃H; more preferably, R¹ is selected from the group consisting of hydrogen, C₃₋₈cycloalkylamino, amino, C₁₋₆alkylamino, C₆₋₁₂arylamino, aminocarbonyl, C₁₋₆alkylaminocarbonyl, —N═N—CH═CH—C₆₋₁₀arylene-hydroxyl, and —N═N—C₆₋₁₀arylene-SO₃H; more preferably , R¹ is selected from the group consisting of hydrogen, cyclohexylamino, amino, C₁₋₆alkylamino, phenylamino, halophenylamino, aminocarbonyl, —N═N—CH═CH-phenylene-hydroxyl, and —N═N-phenylene-SO₃H; more preferably, R¹ is selected from the group consisting of hydrogen, cyclohexylamino, amino, C₁₋₆alkylamino, phenylamino, aminocarbonyl, —N═N—CH═CH-phenylene-hydroxyl, and —N═N-phenylene-SO₃H;

R^{2a} is hydrogen or —SO₃H; R^{2b} is hydrogen or —SO₃H; wherein at least one of R^{2a} or R^{2b} is —SO₃H; for example when R^{2a} is —SO₃H; R^{2b} is hydrogen, or when R^{2a} is hydrogen; R^{2b} is —SO₃H;

R³ is selected from the group consisting of hydrogen, halogen, —SO₃H, C₁₋₆alkyl, amino, C₆₋₁₀aryloxy, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂; preferably, R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, and carboxyl; preferably, R³ is selected from the group consisting of hydrogen, halogen, —SO₃H, C₁₋₄alkyl, amino, C₁₋₄alkylamino, C₆₋₁₀arylamino, C₁₋₄alkoxy, C₁₋₄alkoxyC₁₋₆alkyl, C₁₋₄alkoxycarbonyl, and carboxyl; preferably, R³ is selected from the group consisting of hydrogen, halogen, —SO₃H, C₁₋₄alkyl, amino, C₁₋₄alkylamino, phenylamino, C₁₋₄alkoxy, C₁₋₄alkoxyC₁₋₆alkyl, C₁₋₄alkoxycarbonyl, and carboxyl;

R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, and C₁₋₆alkylaminocarbonyl; preferably, R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, and C₁₋₆alkoxyC₁₋₆alkyl; preferably, R⁴ is selected from the group consisting of hydrogen, halogen, amino, —SO₃H, C₁₋₄alkyl, carboxyl, C₁₋₄alkylamino, C₆₋₁₀arylamino, C₁₋₄alkoxy, and C₁₋₄alkoxyC₁₋₆alkyl;

R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably, R⁵ is selected from the group consisting of hydrogen, halogen, amino, nitro, —SO₃H, C₁₋₆alkyl, hydroxyl, carboxyl, C₁₋₆alkoxy, C₁₋₆alkoxyC₁₋₆alkyl, aminocarbonyl, and C₁₋₆alkylaminocarbonyl; preferably, R⁵ is selected from the group consisting of hydrogen, amino, nitro, —SO₃H, C₁₋₆alkyl, hydroxyl, carboxyl, C₁₋₆alkoxy, C₁₋₆alkoxyC₁₋₆alkyl, aminocarbonyl, and C₁₋₆alkylaminocarbonyl; preferably, R⁵ is selected from the group consisting of hydrogen, halogen, amino, nitro, —SO₃H, C₁₋₄alkyl, hydroxyl, carboxyl, C₁₋₄alkoxy, C₁₋₄alkoxyC₁₋₄alkyl, aminocarbonyl, and C₁₋₄alkylaminocarbonyl; preferably, R⁵ is selected from the group consisting of hydrogen, halogen, amino, nitro, —SO₃H, C₁₋₄alkyl, hydroxyl, carboxyl, C₁₋₄alkoxy, and C₁₋₄alkoxyC₁₋₄alkyl; preferably, R⁵ is selected from the group consisting of hydrogen, amino, nitro, —SO₃H, C₁₋₄alkyl, hydroxyl, carboxyl, C₁₋₄alkoxy, and C₁₋₄alkoxyC₁₋₄alkyl;

R⁶ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably, R⁶ is selected from the group consisting of hydrogen, halogen, amino, C₁₋₆alkyl, C₁₋₆alkoxy, and aminocarbonyl; preferably, R⁶ is selected from the group consisting of hydrogen, halogen, amino, C₁₋₄alkyl, and C₁₋₄alkoxy; preferably, R⁶ is selected from the group consisting of hydrogen, halogen, C₁₋₄alkyl, and C₁₋₄alkoxy; more preferably R⁶ is hydrogen;

R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, carboxyl, —C(OH)═CH—OH, and —NH-phenylene-SO₂—CH═CH₂; preferably, R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, carboxyl, —C(OH)═CH—OH, and aminocarbonyl; preferably R⁷ is selected from the group consisting of hydrogen, halogen, amino, C₁₋₆alkyl, C₁₋₆alkylamino, C₁₋₆alkoxy, C₁₋₆alkoxyC₁₋₆alkyl, carboxyl, —C(OH)═CH—OH, and aminocarbonyl; preferably R⁷ is selected from the group consisting of hydrogen, halogen, amino, C₁₋₄alkyl, C₁₋₄alkylamino, C₁₋₄alkoxy, C₁₋₄alkoxyC₁₋₆alkyl, carboxyl, —C(OH)═CH—OH, and aminocarbonyl; more preferably R⁷ is selected from the group consisting of hydrogen, halogen, amino, C₁₋₄alkyl, carboxyl, —C(OH)═CH—OH, and C₁₋₄alkoxy; more preferably R⁷ is hydrogen, halogen, carboxyl or —C(OH)═CH—OH;

R⁸ is hydrogen or —SO₃H;

R⁹ is hydrogen or —SO₃H;

preferably at least one of R⁸ and R⁹ is —SO₃H; for example when R⁸ is —SO₃H; R⁹ is hydrogen, or when R⁸ is hydrogen; R⁹ is —SO₃H;

R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; preferably, R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, and aminocarbonyl; preferably, R¹⁰ is selected from the group consisting of hydrogen, halogen, C₁₋₄alkyl, amino, hydroxyl, C₁₋₄alkylamino, C₄₋₆cycloalkylamino, phenylamino, C₁₋₄alkoxy, and aminocarbonyl; preferably, R¹⁰ is selected from the group consisting of hydrogen, halogen, C₁₋₄alkyl, amino, hydroxyl, C₁₋₄alkylamino, C₄₋₆cycloalkylamino, and phenylamino; and

R¹¹ is selected from the group consisting of hydrogen, C₁₋₆alkyl, amino, halogen, hydroxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably, R¹¹ is selected from the group consisting of hydrogen, C₁₋₆alkyl, amino, halogen, hydroxyl, C₁₋₆alkylamino, and C₁₋₆alkoxy; preferably, R¹¹ is selected from the group consisting of hydrogen, C₁₋₄alkyl, amino, halogen, hydroxyl, C₁₋₄alkylamino, and C₁₋₄alkoxy; preferably, R¹¹ is selected from the group consisting of hydrogen, C₁₋₄alkyl, halogen, and C₁₋₄alkoxy; more preferably, R¹¹ is hydrogen.

As used in the foregoing and hereinafter, the following definitions apply unless otherwise noted.

The term “salt thereof” as used encompasses the fact that compounds of Formula (I) or (II) contain an acidic proton which can also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “quaternary amine” as used hereinbefore defines the quaternary ammonium salts which the compounds of Formula (I) or (II) are able to form by reaction between a basic nitrogen of a compound of Formula (I) or (II) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen.

The term halo or halogen is generic to fluoro, chloro, bromo and iodo.

As used herein “C₁₋₆alkyl”, as a group or part of a group, defines straight or branched chain saturated hydrocarbon radicals having from 1 to 6 carbon atoms such as for example methyl, ethyl, prop-1-yl, prop-2-yl, but-1-yl, but-2-yl, isobutyl, 2-methyl-prop-1-yl; pent-1-yl, pent-2-yl, pent-3-yl, hex-1-yl, hex-2-yl, 2-methylbut-1-yl, 2-methylpent-1-yl, 2-ethylbut-1-yl, 3-methylpent-2-yl, and the like. Of interest amongst C₁₋₆alkyl is C₁₋₄alkyl.

Where C₁₋₆alkyl groups as defined are divalent, i.e., with two single bonds for attachment to two other groups, they are termed “C₁₋₆alkylene” groups. Non-limiting examples of alkylene groups includes methylene, ethylene, methylmethylene, propylene, ethylethylene, 1,2-dimethylethylene, and the like.

The term “C₃₋₈cycloalkyl”, as a group or part of a group, is a cyclic alkyl group, that is to say, a monovalent, saturated hydrocarbyl group comprising from 3 to 8 carbon atoms and having 1, 2 or 3 cyclic structure. Examples of C₃₋₈cycloalkyl groups include but are not limited to cyclopropyl, cyclobutyl, cyclopentyl, cyclohexyl, cycloheptyl, cyclooctyl. When the suffix “ene” is used in conjunction with a cyclic group, this is intended to mean the cyclic group as defined herein having two single bonds as points of attachment to other groups.

The term “C₆₋₁₂aryl”, as a group or part of a group, refers to a polyunsaturated, aromatic hydrocarbyl group having a single ring (i.e. phenyl) or multiple aromatic rings fused together (e.g. naphthalene), or linked covalently, typically containing 6 to 12 atoms; wherein at least one ring is aromatic. Non-limiting examples of C₆₋₁₂aryl comprise phenyl, biphenylyl, biphenylenyl, or 1- or 2-naphthanelyl.

Where C₆₋₁₂aryl groups as defined are divalent, i.e., with two single bonds for attachment to two other groups, they are termed “C₁₋₆arylene” groups. Non-limiting examples of arylene groups includes phenylene and the like.

The term “amino” refers to the group —NH₂.

The term “C₁₋₆alkylamino”, as a group or part of a group, refers to a group of formula —N(R^{a})(R^{b}) wherein R^{a} is hydrogen or C₁₋₆alkyl as defined above, and R^{b} is C₁₋₆alkyl as defined above. C₁₋₆alkylamino include mono-C₁₋₆alkylamino group such as methylamino and ethylamino and di-C₁₋₆alkylamino group such as dimethylamino and diethylamino. Non-limiting examples of C₁₋₆alkylamino groups include methylamino (NHCH₃), ethylamino (NHCH₂CH₃), n-propylamino, isopropylamino, n-butylamino, isobutylamino, sec-butylamino, tert-butylamino, pentylamino, n-hexylamino, dimethylamino, diethylamino, di-n-propylamino, diisopropylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-t-butylamino, di-i-butylamino, methylpentylamino, methylhexylamino, ethylpentylamino, ethylhexylamino, propylpentylamino, propylhexylamino, and the like.

The term “C₃₋₈cycloalkylamino”, as a group or part of a group, refers to a group of formula —N(R^{g})(R^{h}) wherein R^{g} is hydrogen, C₃₋₈cycloalkyl or C₁₋₆alkyl as defined above, and R^{h} is C₃₋₈cycloalkyl as defined above.

The term “C₆₋₁₂arylamino”, as a group or part of a group, refers to a group of formula —N(R^{d})(R^{c}) wherein R^{d} is hydrogen, C₆₋₁₂aryl or C₁₋₆alkyl as defined above, and R^{c} is C₆₋₁₂aryl as defined above.

The term “C₁₋₆alkoxy” or “C₁₋₆alkyloxy”, as a group or part of a group, refers to a radical having the Formula —OR^{b} wherein R^{b} is C₁₋₆alkyl as defined above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “C₁₋₆alkoxyC₁₋₆alkyl” or “C₁₋₆alkyloxyC₁₋₆alkyl”, as a group or part of a group, refers to a radical having the Formula —R^{e}—OR^{b} wherein R^{e} is a C₁₋₆alkylene, and R^{b} is C₁₋₆alkyl as defined above.

The term “C₁₋₆alkoxycarbonyl” or “C₁₋₆alkyloxycarbonyl”, as a group or part of a group, refers to a group of formula —C(═O)—O—R^{b}, wherein R^{b} is C₁₋₆alkyl as defined above.

The term “aminocarbonyl” refers to the group —(C═O)—NH₂.

The term “C₁₋₆alkylaminocarbonyl”, as a group or part of a group, refers to a group −(C═O)—NR^{a}R^{b} wherein R^{a} is hydrogen or C₁₋₆alkyl as defined above, and R^{b} is C₁₋₆alkyl as defined above.

The term “C₆₋₁₂arylaminocarbonyl”, as a group or part of a group, refers to a group of formula —C(═O)—N(R^{d})(R^{c}) wherein R^{d} is hydrogen, C₆₋₁₂aryl or C₁₋₆alkyl as defined above, and R^{c} is C₆₋₁₂aryl as defined above.

The term “carboxy” or “carboxyl”, as a group or part of a group, refers to the group —CO₂H.

The term “haloC₆₋₁₂aryl”, as a group or part of a group, refers to a C₆₋₁₂aryl radical having the meaning as defined above wherein one or more hydrogens are replaced with one or more halogens as defined above.

It should be noted that the group positions on any molecular moiety used in the definitions may be anywhere on such moiety as long as it is chemically stable.

Groups used in the definitions of the variables include all possible isomers unless otherwise indicated. For instance naphthalenyl includes naphthalen-1-yl and naphthalen-2-yl.

When any variable occurs more than one time in any constituent, each definition is independent.

As used in the specification and the appended claims, the singular forms “a”, “an”, and “the” include plural referents unless the context clearly dictates otherwise. By way of example, “a compound” means one compound or more than one compound.

Some of the compounds of formula (I) may exist in their tautomeric form. Such forms although not explicitly indicated in the above formula are intended to be included within the scope of the present invention.

Preferred features of the compounds of this invention are now set forth.

According to an embodiment, the present invention provides compounds of Formula (I) or (II), wherein at least one of R⁸ and R⁹ is —SO₃H, and R¹, R^{2a}, R^{2b}, R³, R⁴, R⁵, R⁷, R¹⁰, R¹¹ have the same meaning as defined herein.

According to an embodiment, the present invention provides compounds of Formula (I) or (II), wherein R^{2a} is —SO₃H; and R^{2b} is hydrogen and R¹, R³, R⁴, R⁵, R³, R⁷, R⁸, R⁹, R¹⁰, R¹¹ have the same meaning as defined herein.

According to an embodiment, the present invention provides compounds of Formula (I) or (II), wherein R⁸ is —SO₃H; and R⁹ is hydrogen, and R¹, R^{2a}, R^{2b}, R³, R⁴, R⁵, R³, R⁷, R¹⁰, R¹¹ have the same meaning as defined herein.

According to an embodiment, the present invention provides compounds of Formula (I) or (II), wherein R^{2a} is —SO₃H; and R^{2b} is hydrogen and wherein R⁸ is —SO₃H; and R⁹ is hydrogen and R¹, R³, R⁴, R⁵, R³, R⁷, R¹⁰, R¹¹ have the same meaning as that defined herein.

According to an embodiment, the present invention provides compounds of Formula (I) or (II), wherein R^{2a} is hydrogen; and R^{2b} is —SO₃H, and R¹, R³, R⁴, R⁵, R³, R⁷, R⁸, R¹⁰, R¹¹ have the same meaning as that defined herein.

According to an embodiment, the present invention provides compounds of Formula (I) or (II), wherein R⁸ is hydrogen; and R⁹ is —SO₃H, and R¹, R^{2a}, R^{2b}, R³, R⁴, R⁵, R³, R⁷, R¹⁰, R¹¹ have the same meaning as that defined herein.

According to an embodiment, the present invention provides compounds of Formula (I) or (II), wherein R^{2a} is hydrogen; and R^{2b} is —SO₃H and wherein R⁸ is hydrogen; and R⁹ is —SO₃H and R¹, R³, R⁴, R⁵, R³, R⁷, R¹⁰, R¹¹ have the same meaning as that defined herein.

According to an embodiment, the present invention provides compounds of Formula (I) or (II), wherein R¹⁰ is selected from the group consisting of C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl, and —N═N—C₆₋₁₂arylene-SO₃H, and R¹, R^{2a}, R^{2b}, R³, R⁴, R⁵, R³, R⁷, R⁸, R⁹, R¹¹ have the same meaning as that defined herein.

According to an embodiment, the present invention provides compounds of Formula (I) or (II), wherein R¹¹ is hydrogen and R¹, R^{2a}, R^{2b}, R³, R⁴, R⁵, R³, R⁷, R⁸, R⁹, R¹⁰ have the same meaning as that defined herein.

According to an embodiment, the present invention provides compounds of Formula (I) or (II), wherein R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—C₆₋₁₂arylene-SO₃H, and —SO₂—CH═CH₂; preferably R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, and —N═N—C₆₋₁₂arylene-SO₃H;

R^{2a} is hydrogen or —SO₃H;

R^{2b} is hydrogen or —SO₃H;

wherein at least one of R^{2a} or R^{2b} is —SO₃H;

R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂; preferably R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, and C₁₋₆alkylaminocarbonyl;

R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably R⁵ is selected from the group consisting of hydrogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁶ is selected from the group consisting of hydrogen, halogen, amino, and C₁₋₆alkyl;

R⁷ is selected from the group consisting of hydrogen, halogen, amino, carboxyl, —C(OH)═CH—OH, and C₁₋₆alkyl;

R⁸ is hydrogen or —SO₃H;

R⁹ is hydrogen or —SO₃H; wherein at least one of R⁸ or R⁹ is —SO₃H;

R¹⁰ is selected from the group consisting of hydrogen, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and

R¹¹ is hydrogen.

According to an embodiment, the present invention provides compounds of Formula (I) or (II), wherein R⁶ is hydrogen.

According to an embodiment, the present invention provides compounds of Formula (I) or (II), wherein R¹ is selected from the group consisting of hydrogen, C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl, and —N═N—C₆₋₁₂arylene-SO₃H; preferably hydrogen, cyclopropylamino, cyclobutylamino, cyclopentylamino, cyclohexylamino, amino, C₁₋₄alkylamino, phenylamino, naphthalenylamino, halophenylamino, —N═N—CH═CH-phenylenyl-hydroxyl, and —N═N-phenylenyl-SO₃H; and R^{2a}, R^{2b}, R³, R⁴, R⁵, R³, R⁷, R⁸, R⁹, R¹⁰, R¹¹ have the same meaning as that defined herein.

According to an embodiment, the present invention provides compounds of Formula (I) or (II), wherein R¹ is selected from the group consisting of hydrogen, C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl and —N═N—C₆₋₁₂arylene-SO₃H;

R^{2a} is —SO₃H; and R^{2b} is hydrogen;

R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, and carboxyl;

R⁴ is selected from the group consisting of hydrogen, —SO₃H, C₁₋₆alkyl, amino, halogen, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁵ is selected from the group consisting of hydrogen, halogen, —SO₃H, C₁₋₆alkyl, amino, nitro, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably R⁵ is selected from the group consisting of hydrogen, —SO₃H, C₁₋₆alkyl, amino, nitro, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁶ is hydrogen;

R⁷ is hydrogen, carboxyl or —C(OH)═CH—OH;

R⁸ is hydrogen or —SO₃H;

R⁹ is hydrogen or —SO₃H;

R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and

R¹¹ is hydrogen.

According to an embodiment, the present invention provides compounds of Formula (I) wherein

R¹ is selected from the group consisting of hydrogen, C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl, and —N═N—C₆₋₁₂arylene-SO₃H;

R^{2a} is hydrogen or —SO₃H;

R^{2b} is hydrogen or —SO₃H;

wherein at least one of R^{2a} or R^{2b} is —SO₃H;

R³ is selected from the group consisting of hydrogen, —SO₃H, C₁₋₆alkyl, amino, halogen, C₁₋₆alkylamino, C₆₋₁₂arylamino, C₁₋₆alkoxy, and carboxyl;

R⁴ is selected from the group consisting of hydrogen, —SO₃H, C₁₋₆alkyl, amino, halogen, carboxyl, C₁₋₆alkylamino, C₆₋₁₂arylamino, and C₁₋₆alkoxy;

R⁵ is selected from the group consisting of hydrogen, halogen, —SO₃H, C₁₋₆alkyl, amino, nitro, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl; preferably R⁵ is selected from the group consisting of hydrogen, —SO₃H, C₁₋₆alkyl, amino, nitro, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;

R⁶ is hydrogen; and

R⁷ is hydrogen, carboxyl, or —C(OH)═CH—OH.

According to an embodiment, the present invention provides compounds of Formula (II), wherein

R¹ is selected from the group consisting of hydrogen, C₃₋₈cycloalkylamino, amino, C₁₋₆alkylamino, C₆₋₁₂arylamino, haloC₆₋₁₂arylamino, and —N═N—C₆₋₁₂arylene-SO₃H;

R^{2a} is hydrogen or —SO₃H;

R^{2b} is hydrogen or —SO₃H;

wherein at least one of R^{2a} or R^{2b} is —SO₃H;

R⁸ is hydrogen or —SO₃H;

R⁹ is hydrogen or —SO₃H;

R¹⁰ is selected from the group consisting of hydrogen, amino, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and

R¹¹ is hydrogen.

The present invention also encompasses the compounds listed in Table 3, tautomers, salts or ammonium salts thereof.

The present invention also includes processes for the preparation of compounds of the invention.

The compounds of this invention can be prepared as described hereunder. They are generally prepared from starting materials which are either commercially available or prepared by standard means obvious to those skilled in the art. The compounds of this invention can be also prepared using standard synthetic processes commonly used by those skilled in the art of organic chemistry.

Compounds of Formula (I) or (II) can be prepared as illustrated in Schemes 1 or 2 respectively by reacting the amino moiety of a compound of formula (Ia) with the amino moieties of a compound of Formula (Ib) or (IIb) respectively,

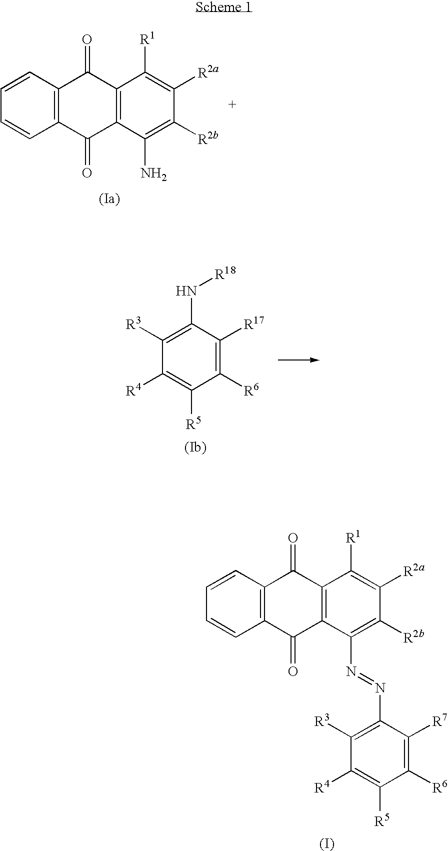

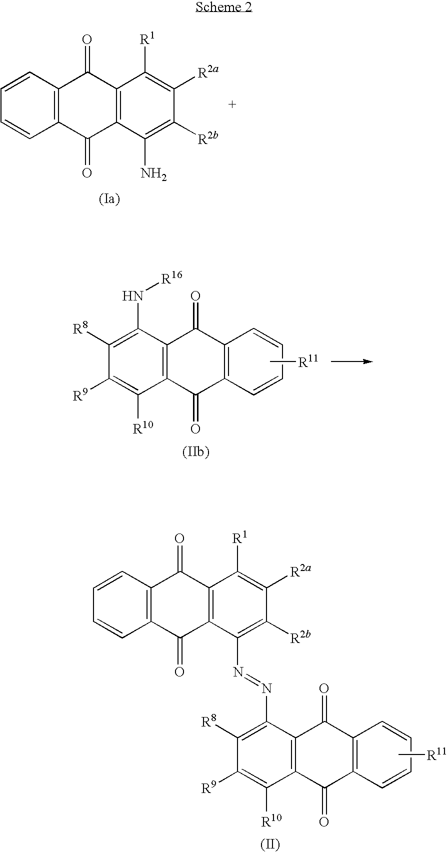

wherein R¹⁶ is selected from the group comprising hydrogen, C₁₋₆cycloalkyl, C₆₋₁₀aryl, haloC₆₋₁₀aryl, 9,10-dioxoanthracenyl, or 4-p-tolyloxysulfonyloxy-phenyl, preferably R¹⁶ is selected from the group comprising hydrogen, C₁₋₆cycloalkyl, C₆₋₁₀aryl, or haloC₆₋₁₀aryl; more preferably, R¹⁶ is selected from the group comprising hydrogen, C₁₋₆cycloalkyl, or C₆₋₁₀aryl; more preferably, R¹⁶ is selected from the group comprising hydrogen or C₁₋₆cycloalkyl;

R¹⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, carboxyl and —NH-phenylene-SO₂—CH═CH₂; and

R¹⁸ is selected from hydrogen, or C₆₋₁₂aryl; preferably hydrogen or phenyl; or

NH—R¹⁸ and R¹⁷ together with the carbon atom to which they are attached form a 5 membered heteroaryl or heterocyclyl ring, each ring being optionally substituted with one or two substituents selected from C₁₋₆alkylcarbonyloxy or carboxyl, for example NH—R¹⁸ and R¹⁷ together with the carbon atom to which they are attached may form a pyrrolyl, said pyrrolyl being optionally substituted with one or two substituents selected from C₁₋₆alkylcarbonyloxy or carboxyl;

and R¹, R^{2a}, R^{2b}, R³, R⁴, R⁵, R⁶, R⁷, R⁸, R⁹, R¹⁰, R¹¹ have the same meaning as defined herein.

Compounds of Formula (I) can also be prepared by reacting a compound of Formula (Ia) in the presence of an oxidoreductase.

The product may be readily purified from the reaction using the methods described herein.

In the reactions described, it may be necessary to protect reactive functional groups, for example hydroxy, amino, or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1999.

According to the present invention, compounds of Formula (I) or (II) may be may be produced through the use of oxidoreductases, for example produced by microorganisms. As compared to “whole cells” process using microorganisms which takes several days (for example three days) to synthesize the azo bond, the present process has the advantage of using isolated enzyme. This enzymatic process allowed reducing the reaction time to a few hours and is therefore an advantage. Since enzymes are produced by microorganisms (that can grow on wastes), the catalyst is therefore renewable, in contrast to traditional chemical catalysts.

According to an embodiment, the oxidoreductase enzyme suitable for use in said process is selected from the group consisting of laccase, peroxidase, cellobiose dehydrogenase, and tyrosinase.

In a preferred embodiment, laccase are used. Laccases show high potential as industrial biocatalysts. Laccases (EC 1.10.3.2) are benzenediol:oxygen oxidoreductases that oxidise a wide variety of organic compounds, causing O- and N-demethylation reactions, carbon-carbon bond cleavage or polymerizations (Burton S. Current organic chemistry 2003; 1317-1331). These biocatalysts require molecular oxygen as the electron acceptor therefore producing water (Solomon E I. et al Chemical Review 1996, 96 (17) 2563-2606). The use of this enzyme therefore represents an advantage for industrial applications. Moreover, laccases can be cheaply available as they may be secreted by some fungi in high level upon induction.

According to an embodiment, the laccase used in the present process is produced by white rot fungi.

The reaction mixture comprise a quantity of laccase either in solution or immobilized so that the resulting activity of the enzyme in the solution is equal to or greater than 1,10, 35, 50, 100, 150, 200, 250, 300, 400, 500, 600, 700, 800, 900, 1000, 1100, 1150, 1200, 1250, 1300, 1400, 1500, 1600, 1700, 1800, 1900, or 2000 U.L⁻¹ or a value in the range between any two of the aforementioned values, preferably between 1 and 1000 U.L⁻¹. Preferably the laccase is used in a quantity of at least 10 U.L⁻¹, preferably above 10 U.L⁻¹, for example of at least 20 U.L⁻¹, of at least 30 U.L⁻¹, of at least 40 U.L⁻¹, more preferably of at least 50 U.L⁻¹, preferably of at least 60 U.L⁻¹, preferably of at least 70 U.L⁻¹, more preferably of at least 80 U.L⁻¹, yet more preferably of at least 90 U.L⁻¹, yet more preferably of at least 100 U.L⁻¹.

Preferably, the enzyme for use in said process can be used as a free enzyme or as an immobilized enzyme. In an embodiment said enzyme is immobilized on a solid support. In an embodiment, said process is performed using immobilized enzyme with or without support. Suitable solid supports can be selected from the non-limiting group comprising glass beads, perlite, montmorillonite, alginate and carrageenan. Preferably said solid support is perlite.

According to an embodiment, the coupling reaction is performed in a bioreactor.

The reaction is preferably performed aerobically using common glassware (e.g. beaker or flask) agitated by magnetic stirrer, or by agitation in shaking flasks or using a fermentor, if appropriate with the introduction of air or oxygen. In an embodiment, the process also comprises the step of at least sequentially agitating or aerating, or oxygenating the reaction mixture or performing the reaction under overpressure of oxygen.

In an embodiment, said reaction is performed under overpressure of oxygen, more preferably at about 2 atmospheric bars.

According to an embodiment, the reaction is performed at a pH between about 3 and 9, preferably at a pH between about 4 and 8, more preferably at a pH between about 4 and 7, yet more preferably at a pH between about 4 and 6, yet more preferably at a pH between about 4 and 5, preferably at pH of about 4.5. According to an embodiment, the reaction is performed in a buffer selected from the group comprising tartaric buffer, acetate buffer, phosphate borate buffer. Preferably the reaction is performed in tartaric buffer.

The enzymatic reaction can be performed at temperatures ranging from about 15-60° C., preferably about 20-50° C., preferably about 20-40° C., yet more preferably about 20-30° C. In a preferred embodiment, the enzymatic reaction is performed at room temperature. Downstream processing can be through classical processes, including, but not limited to salting out, lyophilization, spray drying, air drying and the like.

The course of the reaction can be monitored with the aid of the pH measurement of the medium, the aid of HPLC method, spectrophotometric method or by thin layer chromatography. The compounds of interest are isolated from the reaction mixture by known methods, taking into account the chemical, physical and biological properties of the products.

The skilled person will appreciate that reaction products and unreacted reagents may be detected by thin layer chromatography on silica gel with polar solvent mixture, for example n-butanol/acetic acid/water or methylethylketone/acetone/water as the mobile phase. Alternatively the detection can be carried out by known liquid chromatographic methods (e.g. HPLC), and by mass spectrometry.

The compound can be further purified by appropriate filtration through a plug of reversed phase silica gel with water as the mobile phase up to a purity of about 90%. Further purification can be accomplished by known methods, for example, using semi-preparative HPLC.

The compounds of Formula (I) or (II) are particularly convenient to dye textile, leather, hair and other articles. The compounds are particularly useful as red dyes.

Also part of the invention, is the use of the compounds of the present invention as a dye for dyeing natural or synthetic materials such as paper, cellulose, polyamide, hair, leather or glass fibers.

The present invention therefore encompasses a method of dyeing an article or a substrate comprising the step of contacting said article or substrate with a compound of Formula (I) or (II) according to the invention.

Suitable articles for this dyeing method can be made of a material selected from a fabric, yarn, fiber, garment or film made of a material selected from the group consisting of fur, hair, hide, leather, silk, wool, cationic polysaccharide, synthetic polyamide, and the like.

The dye according to the invention particularly provides an even coloration on acetate, cotton, nylon, PES, acrylic and wool keratin, fibers with favorable dyeing properties such as good fastness to light, washing, rubbing and perspiration.

The present compounds according to the invention are particularly suitable for dyeing organic materials, such as keratin-containing fibers, wool, leather, silk, cellulose or polyamides, cotton or nylon, and preferably human hair.

The dyeings obtained are distinguished by their depth of shade and their good fastness properties to washing, such as, for example, fastness to light, shampooing and rubbing. The stability, in particular the storage stability of the dyes according to the invention are excellent.

The present invention also encompasses formulation containing at least one compound of Formula (I) or (II).

Formulations, containing dyes according to the invention of the structural Formula (I) or (II) provide an even coloration for example on keratin fibers, in particular human hair, with favorable dyeing properties such as good fastness to light, washing, rubbing and perspiration. Natural colorations with low selectivity can be obtained already under gentle conditions.

The dyes of the structural Formula (I) or (II) according to the invention are preferably present in the formulation according to the invention in a total amount of from about 0.01 to 10% by weight, preferablyfrom about 0.1 to 8% by weight, more preferably from 0.1 to 7% by weight, preferably from 0.3 to 6% by weight, preferably from 0.4 to 5% by weight, in particular 0.5 to 4% by weight.

To produce special color shades, besides the dyes of the structural Formula (I) or (II) according to the invention, it is possible to add to the compounds according to the invention or to the formulation according to the invention, one or more additional customary direct dye from the group consisting of acidic dyes, basic dyes, nitro dyes, azo dyes, anthraquinone dyes and triphenylmethane dyes. In addition, the formulations according to the invention can also comprise naturally occurring dyes, such as, for example, henna red, henna neutral, henna black, camomile, sandalwood, black tea, buckthorn bark, sage, logwood, madder root, catechu, sedre and alkanna root.

The abovementioned additional direct dyes and naturally occurring dyes may be present in a total amount of from about 0.01 to 5% by weight, the total content of dyes in the formulations according to the invention being preferably from about 0.01 to 10% by weight, in particular about 0.1 to 5% by weight.

The present invention also covers articles or substrate dyed with a compound of Formula (I) or (II).

The following examples are intended to illustrate the present invention and not to limit it thereto.

### Examples

### Example 1

### Synthesis of Compounds of Formula (I) or (II)

Compounds of Formula (I) or (II) may be prepared according to any of the protocol N to T.

The compounds may be produced by the reaction of two precursors listed in Table 1 or by the reaction of a precursor listed in Table 1 with a precursor listed in Table 2. Said reaction is preferably performed in the presence of enzymes, in particular oxidoreductase enzyme. Suitable oxidoreductase used for the preparation process can be selected from the group consisting of laccases, peroxidases, cellobiose deshydrogenases and tyrosinases, and preferably laccases.

Unless indicated otherwise, All reagents used were either obtained commercially or were prepared in a manner known per se.

Unless indicated otherwise, the enzyme activity was determined according to protocols A or B.

Protocol A:

Laccase activity was determined by oxidation of ABTS [2′,2-azino-bis-(3-ethylbenzo thiazoline-6-sulfonic acid)] (4 mM) (Sigma-Aldrich) into a stable cationic radical ABTS^{·+} (absorption coefficient, ε_{M}=34 220 M⁻¹.cm⁻¹) in tartaric buffer (100 mM) at pH 4.5. One unit was defined as the amount of enzyme that oxidizes 1 μmol of ABTS per minute. The increase in absorbance at 414 nm was monitored during about 2 min with a Beckman DU-800 spectrophotometer connected to a high performance temperature controller (Analis, Namur, Belgium), which maintained the temperature of the reaction mixture at 25° C.

Protocol B:

Cellobiose dehydrogenase activity was assessed using a modified protocol from Baminger et al. J Microbiol Meth 1999. 35:253-9. Lactose was replaced by cellobiose. One unit of enzyme activity was defined as the amount of enzyme reducing 1 μmol of DCIP (2,6-dichlorophenol-indolophenol)/min.

Unless indicated otherwise, the biocatalytic material was prepared according to protocol C.

Protocol C:

Biocatalytic material was prepared as following. Perlite Perlagri 40 was firstly silanized by immersion in acetone containing aminopropyltrimethoxysilane (APTES—4%) during 24 h at 45° C. under stirring. The supernatant was removed and carrier was dried at 45° C. during 24 h. Carrier was activated by immersion in phosphate buffer containing 5% glutaraldehyde under magnetic stirring during 2 h at 4° C. After washing, the carrier was filtered on Duran n^{o}4 filter and 1 L of water containing Enzymatic powder (125 mg) was mixed with activated carrier. The immobilized enzyme was finally stored at 4° C. in acidic water pH 4.5 before being packed in a column.

Unless indicated otherwise, the bioreactor was prepared according to protocol D.

Protocol D:

The biocatalytic material was rinsed with acid water (pH 4.5, HCl) and was used to fill the enzymatic bioreactor (Minilab, Wetlands, Belgium), following the instructions of the manufacturer.

Unless indicated otherwise, the reactions were followed up according to protocol E.

Protocol E: Spectrophotometric Follow Up of Biotransformation.

Sampling were made at interval times and scanning was realized between 300 and 700 nm with a Beckman DU-800 spectrophotometer connected to a high performance temperature controller (Analis, Namur, Belgium) at 25° C. The decrease in absorbance at the maximum wavelength was monitored. Increase at maximal wavelength of the product was also monitored.

Unless indicated otherwise, the biotransformation monitoring and compound production, isolation and/or purification were performed according to protocol F, G, H I, and/or J.

Protocol F:

Monitoring of the biotransformation was made through thin layer chromatography, using pre-coated TLC plates SIL G-25 (Macherey Nagel) with methylethylketone/acetone/water (80/20/80:v/v/v) as eluant.

Protocol G:

Capillary electrophoresis analysis was carried out with a P/ACE MDQ Beckman Coulter equipped with a photodiode array UV/Vis detector controlled by a P/ACE station software (Analis, Gent, Belgium). The fused-silica capillary (Analis, Gent, Belgium) (50 cm 75 lm ID) was maintained in a cartridge with a detection window of 100·800 lm. The separation voltage was 25 kV. The procedure consisted of an initial 1 min wash with NaOH 0.1 M under 138 kPa followed by a 1 min wash with the initiator (provided by Analis) under 138 kPa. The capillary was then flushed with the buffer MEKC 9.2 during 2 min at 138 kPa. When a constant run current was achieved, the sample was injected by hydrodynamic injection for 5 s under 3.5 kPa, followed by injection of water during 10 s under 0.7 kPa. A 20 min separation step was performed under a 25 kV potential. After the completion of the procedure, the capillary was rinsed with NaOH 0.1 M during 1 min under 138 kPa. The absorbance from 190 to 600 nm was monitored with an on-column photodiode array detector and acquisition of the electrophoregram was performed at 190 nm.

Protocol H:

HPLC follow up was performed with a system comprising a Waters pump, a Waters 996 photodiode array detector (for analytical separations) or a Waters 486 absorbance detector (for semi-preparative HPLC) (Waters, Milford, Mass., USA). Analytical separations were performed with a Waters Novapack C-18 column (4.6·250 mm). The mobile phase was acetonitrile/water (10/90 of analytical grade). The flow rate was 0.3 ml min_{—}1. Samples were filtered and injected. Detection was performed at 220 nm and on-line UV-Vis absorbance scans were performed.

Protocol I:

Semi-preparative HPLC was performed with a Waters Novapack C-18 column (22·250 mm) (Waters, Milford, Mass., USA). The mobile phase was acetonitrile as eluent A and water as eluent B. The gradient applied was 10/90 (A/B: v/v) over 10 min, 15/85 (A/B: v/v) over 10 min, 20/80 (A/B: v/v) over 10 min and 25/75 (A/B: v/v) over 10 min. The flow rate was 20 ml min_{—}1. On-line UV-Vis absorbance scans were performed.

Protocol J: Dye Recovery

In order to precipitate the dye, samples were treated with 0.9 M NaCl. A centrifugation was then carried out.

Unless otherwise indicated, NMR spectra were determined according to protocol K.

Protocol K:

The 1H and 13C NMR spectra were recorded using a Bruker Avance 500 spectrometer (Bruker, Wissembourg, France) in D2O or MeOD as solvent. Spectra are reported in ppm. Spectra in D2O were obtained with 2,2-dimethyl-2-silapentane-5-sulfonate sodium (DSS) as internal standard.

Unless otherwise indicated, mass spectra were acquired and analyzed according to protocol L1 or L2.

Protocol L1:

ESI/MS. The mass spectra were acquired using a Thermo Finnigan Ion Trap LCQ spectrometer, equipped with an ESI source (Thermo electron, San Jose, Calif., USA). High Resolution Mass Spectrometry (HRMS) analysis was performed at University of Mons-Hainaut (Belgium).

Protocol L2:

ESI/MS. The mass spectra were acquired using a Waters micromass ZMD MC364 spectrometer.

Unless otherwise indicated, the precursors were prepared according to protocol M.

Protocol M:

To a solution of 1-bromaminic acid, sodium salt (0.404 g, 1 mmol) in water/ethanol 9/1 (100 mL) were added the appropriate amine (2 mmol), Na₂CO₃, 10 H₂O (0.645 g, 2.25 mmol) and CuSO₄, 5 H₂O (0.312 g, 1.25 mmol). The mixture was stirred at reflux during 4 hours. After evaporation, the residue was dissolved in H₂O (100-150 mL). NaCl (40 g) was added and the solution was stirred at room temperature during 1-2 h and filtered. The precipitate was purified by column chromatography on RP18 silica gel using a mixture of water/methanol 1/1 as eluent.

Compounds of Formula (I) or (II) may be prepared according to any one of protocols N to T.

Protocol N:

The precursor (62.5 μM) was solubilized in phosphate borate buffer 0.05 M pH 4.5 and biotransformation was carried out in the presence of 10 U/L of Pycnoporus sanguineus laccase. The enzyme activity was determined using protocol A. Reaction was performed at 25° C. during 24 h.

Protocol O:

Precursors (500 μM each) were solubilized in tartaric buffer 0.1 M and the biotransformation was carried out in the presence of 100 U/L of laccase. Enzyme activity was determined using protocol A. Reaction was performed at about 25° C. during about 24 h. Samples were withdrawn and diluted 10 time for spectrophotometric measurement.

Protocol P:

Precursors (875 μM) were solubilized in malt extract 20 g/L. Reaction was carried out at about 25° C. during about 24 h in the presence of Cellobiose dehydrogenase. Enzyme activity was determined using protocol B.

Protocol Q:

Precursors (875 μM) were solubilized in acetate buffer 0.05 M pH 4. Reaction was carried out at about 25° C. during about 24 h in presence of 1 μg/L Tyrosinase (Sigma).

Protocol R:

Precursor was solubilized in water (2.35 mM) and in acetate buffer (50 mM pH 4.5) and a 1 liter volume of precursor was passed through the bioreactor (protocol D) containing 500 U L⁻¹ immobilized lactase (protocol C) at a flow rate of about 100 ml/min (total: 10 min) at 25° C. After the passage, the medium was oxygenated during about 15 min, and then passed again on the bioreactor. The process was repeated about 14 times, with a total contact time between precursor and enzyme of 2 h 20 hours. A maturation phase was obtained by only letting the medium during 24 h at room temperature. Dye was recovered from the medium following protocol J.

Protocol S:

Precursor (11.85 mM) was solubilized in acetate buffer 0.1 M pH 4.5. Biotransformation was carried out in the presence 250 U L⁻¹ laccase. Enzyme activity was determined using protocol A. The reaction was carried out at about 25° C., under agitation.

Protocol T:

Precursor (1.54 mM) was solubilized in tartaric buffer 100 mM pH 4.5. Biotransformation was carried out in the presence of 35 U.l⁻¹ laccase. Enzyme activity was determined using protocol A. Reaction was carried out at about 25° C.

Structural formulas of suitable precursors are listed in Tables 1 and 2.

TABLE 1

| Precursor | Name | Structure |
|---|---|---|
| I1 | 1-Amino-4-cyclohexylamino-9,10- dioxo-9,10-dihydro-anthracene-2- sulfonic acid | 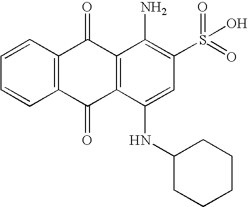 |
| I2 | 1,4-Diamino-anthraquinone | 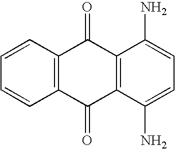 |
| I3 | 1-Amino-9,10-dioxo-9,10-dihydro- anthracene-2-sulfonic acid | 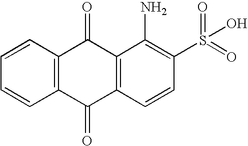 |
| I4 | 1-Amino-9,10-dioxo-4-phenylamino- 9,10-dihydro-anthracene-2-sulfonic acid | 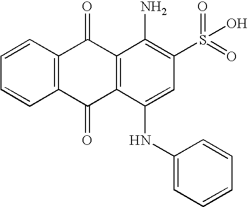 |
| I5 | 1-Amino-4-(4-chloro-phenylamino)- 9,10-dioxo-9,10-dihydro- anthracene-2-sulfonic acid | 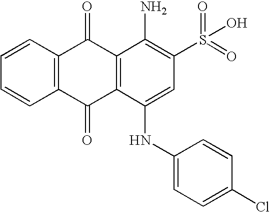 |
| I6 | 1,4-Diamino-9,10-dioxo-9,10- dihydro-anthracene-2-sulfonic acid | 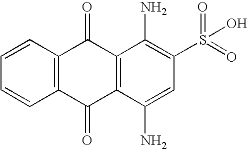 |
| I31 | 1,1′-dianthrimide | 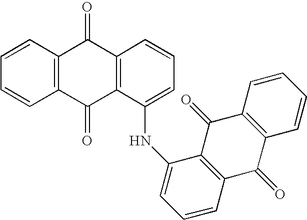 |
| I32 | 1-Amino-9,10-dioxo-4-(4- p-tolyloxysulfonyloxy-phenylamino)- 9,10-dihydro-anthracene-2-sulfonic acid | 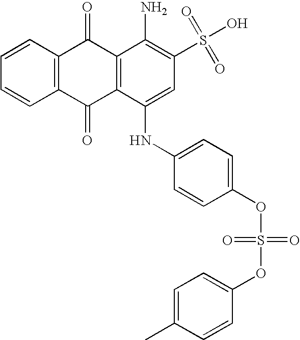 |

TABLE 2

| Precursor | Name | Structure |
|---|---|---|
| I7 | 3-Amino-benzene- sulfonic acid | 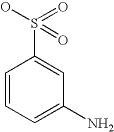 |
| I8 | 2,5-Dimethyl- phenylamine | 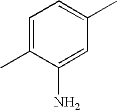 |
| I9 | 1-H-Indole | 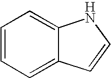 |
| I10 | 4-Amino-benzene- sulfonic acid | 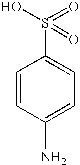 |
| I11 | 2.6-Dichloro-4-nitro- phenylamine | 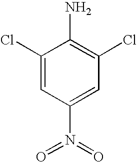 |
| I12 | Acetic acid 1H-indol- 3-yl ester | 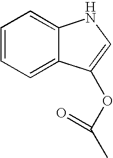 |
| I13 | 4-amino-benzoic acid | 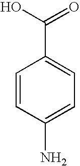 |
| I14 | 4-Methoxy- phenylamine | 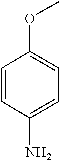 |
| I15 | 2-Amino-5-hydroxy- benzoic acid | 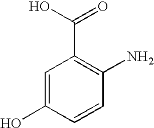 |
| I16 | 2-Amino-3-hydroxy- benzoic acid | 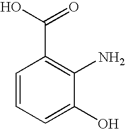 |
| I17 | 2-Amino-3-methoxy- benzoic acid | 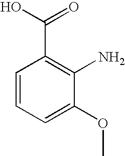 |
| I18 | 3-Amino-2-hydroxy- benzoic acid | 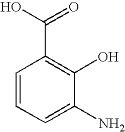 |
| I19 | N-Phenyl-benzene-1,4- diamine | 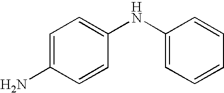 |
| I20 | Benzyl-(9H-purin-6- yl)-amine | 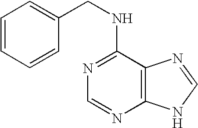 |
| I21 | Diphenyl-amine | 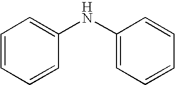 |
| I22 | 2-Methoxy- phenylamine | 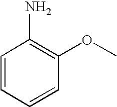 |
| I23 | 2-Methoxy-5-methyl- phenylamine | 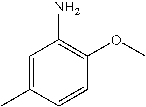 |
| I24 | 2-Amino-benzoic acid | 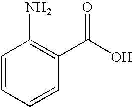 |
| I25 | 2-Methoxy-6-methyl- phenylamine | 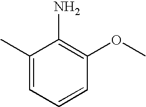 |
| I26 | 5-Fluoro-2-methoxy- phenylamine | 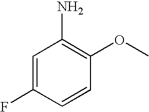 |
| I27 | 2,5-Dimethoxy- phenylamine | 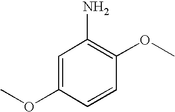 |
| I28 | 3-Amino-4-methoxy- benzoic acid | 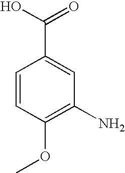 |
| I29 | 3-Amino-4-methoxy- benzoic acid methyl ester | 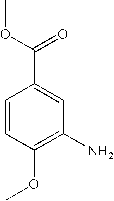 |
| I30 | 2-Phenoxy- phenylamine | 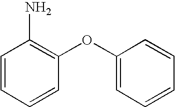 |

### Synthesis of compound 1: 1-[(4-amino-9,10-dioxo-3-sulfo-9,10-dihydroanthracen-1-yl)diazenyl]-4-cyclohexylamino-9,10-dioxo-9,10-dihydroanthracene-2-sulfonic acid, sodium salt

Compound 1 was synthesized, by biotransformation of Precursor I1 following protocol T. biotransformation was analyzed by CE (protocol G), HPLC (protocol H) purified by semi preparative HPLC (protocol I) and analyzed by NMR (protocol K), and ESI (protocol L1).

Results are conformed to the expected structure:

C₃₄H₂₄N₄O₁₀S₂Na₂; Preparative HPLC tR: 31 min; analytical HPLC tR: 10 min; CE tₘ: 13 min; IR (KBr) v 3500, 2931, 1666, 1629, 11593, 1582, 1484, 1402, 1204, 1047, 1021, 732 cm⁻¹; MS (ESI negative mode) m/z (%) 356 (100, [M−2Na]²⁻), 713 (58, [M−2Na+H]⁻), 735 (33, [M−Na]⁻); HRMS (ESI positive mode) m/z found: 715.1165, calc.: 715.1169 (C₃₄H₂₇N₄O₁₀S₂). ¹H NMR δ (500.2 MHz, MeOD) 1.44 (1 H, m), 1.55 (4 H, m), 1.71 (1 H, m), 1.86 (2 H, m), 2.16 (2 H, m), 3.78 (1 H, m), 7.71 (2 H, t), 7.79 (2 H, q), 7.89 (1 H, s), 7.97 (2 H, t), 8.26 (2 H, dd), 8.65 (1 H, s); ¹³C NMR δ (125.8 MHz, MeOD) 25.48, 26.82, 33.93, 52.12, 114.31, 114.44, 118.00, 127.22, 127.48, 127.82, 127.85, 129.41, 132.16, 134.38, 134.52, 134.76, 134.90, 135.35, 135.49, 135.90, 136.19, 143.45, 144.52, 145.91, 150.91, 151.38, 185.40, 185.62, 186.37, 186.55

Alternatively compound 1 was synthesized through protocols N, O, P, Q, R, or S.

The bioconversion of I1 using tyrosinase or CDH (protocols P and Q) was analyzed through TLC (protocol F). rf of the product was 0.38 and was the same as the one obtained for laccase bioconversion (protocol T).

At increased substrate concentration following protocol S a yield of 70% was reached.

In an improved method, protocol C, D and R were combined and allowed, in 2 hour 30 of contact with the biocatalytic material, the production of dye free of enzyme (no risk of skin sensitivity due to the enzyme protein into the dye) as well as the reuse of the biocatalytic material.

### Synthesis of compound 2: 9,10-Dioxo-4-phenylamino-1-phenylazo-9,10-dihydro-anthracene-2-sulfonic acid, sodium salt

Compound 2 was synthesized, by biotransformation of Precursor I4 following protocol N. biotransformation was followed by spectrophotometry (protocol E), TLC (protocol F) and ESI (protocol L1). m/z was 483 in negative mode.

### Synthesis of compound 3: 4-(4-Chloro-phenylamino)-1-(4-chloro-phenylazo)-9,10-dioxo-9,10-dihydro-anthracene-2-sulfonic acid, sodium salt

Compound 3 was synthesized, by biotransformation of I5 (synthesized through protocol M) using protocol N. Biotransformation was followed by spectrophotometry (protocol E) TLC (protocol F) and ESI (protocol L1). m/z was 550 in negative mode.

### Synthesis of compound 4: 4-Cyclohexylamino-9,10-dioxo-1-(3-sulfo-phenylazo)-9,10-dihydro-anthracene-2-sulfonic acid, sodium salt

Compound 4 was synthesized, by biotransformation of precursors I1 and I7 following protocol O. Biotransformation was followed by spectrophotometry (protocol E) and ESI (protocol L1) and TLC (protocol F). m/z was 590 (sodium adduct) in negative mode.

### Synthesis of compound 5: 9,10-Dioxo-1,4-bis-(3-sulfo-phenylazo)-9,10-dihydro-anthracene-2-sulfonic acid, sodium salt

Compound 5 was synthesized, by biotransformation of precursors I1 and I7 following protocol O. Biotransformation was followed by spectrophotometry (protocol E) and ESI (protocol L1) and TLC (protocol F). m/z were 654 (sodium adduct) and 676 (di sodium adduct) in negative mode.

### Synthesis of compound 6: 4-Cyclohexylamino-9,10-dioxo-1-(4-sulfo-phenylazo)-9,10-dihydro-anthracene-2-sulfonic acid, sodium salt

Compound 6 was synthesized, by biotransformation of precursors I1 and I10 following protocol O. Biotransformation was followed by spectrophotometry (protocol E) and ESI (protocol L1) and TLC (protocol F). m/z was 590 (sodium adduct) in negative mode.

### Synthesis of compound 7: 9,10-Dioxo-1,4-bis-(4-sulfo-phenylazo)-9,10-dihydro-anthracene-2-sulfonic acid, sodium salt

Compound 7 was synthesized, by biotransformation of precursors I1 and I10 following protocol O. Biotransformation was followed by spectrophotometry (protocol E) and ESI (protocol L1) and TLC (protocol F). m/z were 654 (sodium adduct) and 676 (di sodium adduct) in negative mode.

### Synthesis of compound 8: 4-Cyclohexylamino-1-[2-((Z)-1,2-dihydroxy-vinyl)-phenylazo]-9,10-dioxo-9,10-dihydro-anthracene-2-sulfonic acid, sodium salt

Compound 8 was synthesized, by biotransformation of precursors I1 and I12 following protocol O. Biotransformation was followed by spectrophotometry (protocol E) and ESI (protocol L1) and TLC (protocol F). m/z was 546 in negative mode.

### Synthesis of compound 9: 4-Cyclohexylamino-9,10-dioxo-1-phenylazo-9,10-dihydro-anthracene-2-sulfonic acid, sodium salt

Compound 9 was synthesized, by biotransformation of precursors I1 and I21 following protocol O. Biotransformation was followed by spectrophotometry (protocol E) and ESI (protocol L1) and TLC (protocol F). m/z was 489 in negative mode.

### Synthesis of compound 10: 4-Cyclohexylamino-1-(2-methoxy-phenylazo)-9,10-dioxo-9,10-dihydro-anthracene-2-sulfonic acid, sodium salt

Compound 10 was synthesized, by biotransformation of precursors I1 and I22 following protocol O. Biotransformation was followed by spectrophotometry (protocol E) and ESI (protocol L2) and TLC (protocol F). m/z was 518.9 in negative mode.

### Synthesis of compound 11: 4-Cyclohexylamino-1-(2-methoxy-5-methyl-phenylazo)-9,10-dioxo-9,10-dihydro-anthracene-2-sulfonic acid, sodium salt

Compound 11 was synthesized, by biotransformation of precursors I1 and I23 following protocol O. Biotransformation was followed by spectrophotometry (protocol E) and ESI (protocol L2) and TLC (protocol F). m/z was 533 in negative mode.

### Synthesis of compound 12: 4-(4-Cyclohexylamino-9,10-dioxo-2-sulfo-9,10-dihydro-anthracen-1-ylazo)-benzoic acid, sodium salt

Compound 12 was synthesized, by biotransformation of precursors I1 and I13 following protocol O. Biotransformation was followed by spectrophotometry (protocol E) and ESI (protocol L2) and TLC (protocol F). m/z was 532 in negative mode.

### Synthesis of compound 13: 2-(4-Cyclohexylamino-9,10-dioxo-2-sulfo-9,10-dihydro-anthracen-1-ylazo)-benzoic acid, sodium salt

Compound 13 was synthesized, by biotransformation of precursors I1 and I24 following protocol O. Biotransformation was followed by spectrophotometry (protocol E) and ESI (protocol L2) and TLC (protocol F). m/z was 532 in negative mode.

### Synthesis of compound 14: 4-Cyclohexylamino-1-(5-fluoro-2-methoxy-phenylazo)-9,10-dioxo-9,10-dihydro-anthracene-2-sulfonic acid, sodium salt

Compound 14 was synthesized, by biotransformation of precursors I1 and I26 following protocol O. Biotransformation was followed by spectrophotometry (protocol E) and ESI (protocol L2) and TLC (protocol F). m/z was 536 in negative mode.

### Synthesis of compound 15: 3-(4-Cyclohexylamino-9,10-dioxo-2-sulfo-9,10-dihydro-anthracen-1-ylazo)-4-methoxy-benzoic acid, sodium salt

Compound 15 was synthesized, by biotransformation of precursors I1 and I28 following protocol O. Biotransformation was followed by spectrophotometry (protocol E) and ESI (protocol L) and TLC (protocol F). m/z was 562 in negative mode.

### Synthesis of compound 16: 3-(4-Cyclohexylamino-9,10-dioxo-2-sulfo-9,10-dihydro-anthracen-1-ylazo)-4-methoxy-benzoic acid methyl ester, sodium salt

Compound 16 was synthesized, by biotransformation of precursors I1 and I29 following protocol O. Biotransformation was followed by spectrophotometry (protocol E) and ESI (protocol L2) and TLC (protocol F). m/z was 576 in negative mode.

### Synthesis of compound 17: 4-Cyclohexylamino-9,10-dioxo-1-(2-phenoxy-phenylazo)-9,10-dihydro-anthracene-2-sulfonic acid, sodium salt

Compound 17 was synthesized, by biotransformation of precursors I1 and I30 following protocol O. Biotransformation was followed by spectrophotometry (protocol E) and ESI (protocol L2) and TLC (protocol F). m/z was 580 in negative mode.

The present invention encompasses compounds as illustrated in Table 3 as well as tautomers, and salts thereof.

TABLE 3

| Compound | Name | structure |
|---|---|---|
| 1 | 1-[(4-amino-9,10-dioxo- 3-sulfo-9,10- dihydroanthracen-1- yl)diazenyl]-4- cyclohexylamino-9,10- dioxo-9,10- dihydroanthracene-2- sulfonic acid | 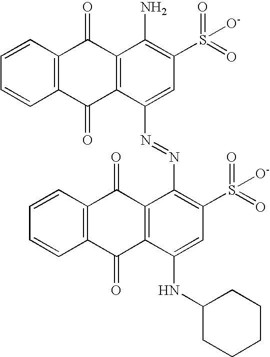 |
| 2 | 9,10-Dioxo-4- phenylamino-1- phenylazo-9,10-dihydro- anthracene-2-sulfonic acid | 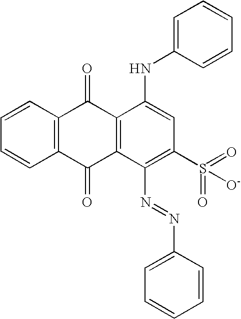 |
| 3 | 4-(4-Chloro- phenylamino)-1-(4- chloro-phenylazo)-9,10- dioxo-9,10-dihydro- anthracene-2-sulfonic acid | 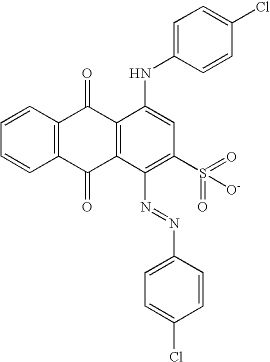 |
| 4 | 4-Cyclohexylamino-9,10- dioxo-1-(3-sulfo- phenylazo)-9,10-dihydro- anthracene-2-sulfonic acid | 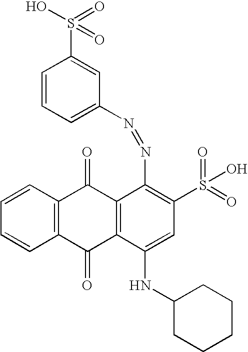 |
| 5 | 9,10-Dioxo-1,4-bis-(3- sulfo-phenylazo)-9,10- dihydro-anthracene-2- sulfonic acid | 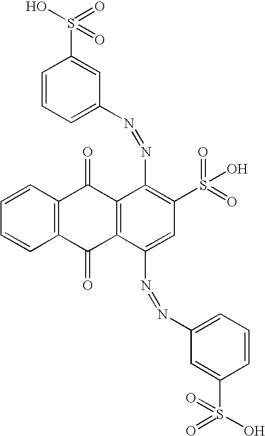 |
| 6 | 4-Cyclohexylamino-9,10- dioxo-1-(4-sulfo- phenylazo)-9,10-dihydro- anthracene-2-sulfonic acid | 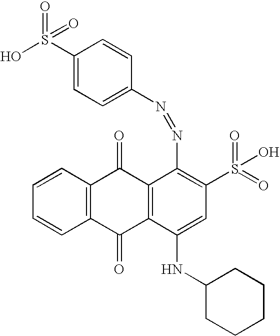 |
| 7 | 9,10-Dioxo-1,4-bis-(4- sulfo-phenylazo)-9,10- dihydro-anthracene-2- sulfonic acid | 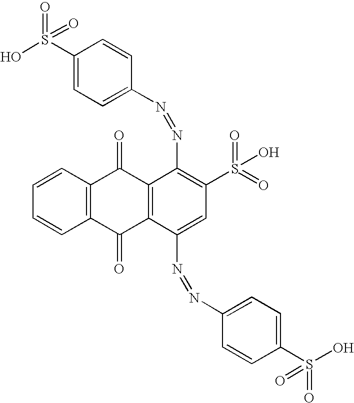 |
| 8 | 4-Cyclohexylamino-1-[2- ((Z)-1,2-dihydroxy-vinyl)- phenylazo]-9,10-dioxo- 9,10-dihydro-anthracene- 2-sulfonic acid | 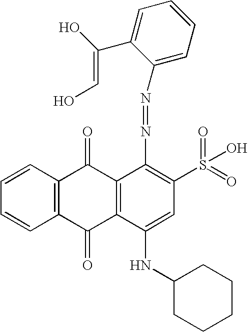 |
| 9 | 4-Cyclohexylamino-9,10- dioxo-1-phenylazo-9,10- dihydro-anthracene-2- sulfonic acid | 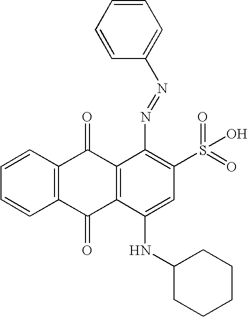 |
| 10 | 4-Cyclohexylamino-1-(2- methoxy-phenylazo)- 9,10-dioxo-9,10-dihydro- anthracene-2-sulfonic acid | 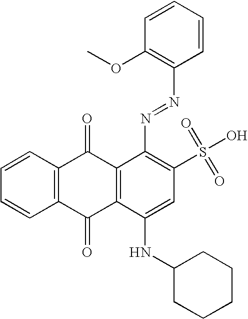 |
| 11 | 4-Cyclohexylamino-1-(2- methoxy-5-methyl- phenylazo)-9,10-dioxo- 9,10-dihydro-anthracene- 2-sulfonic acid | 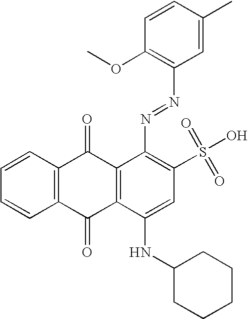 |
| 12 | 4-(4-Cyclohexylamino- 9,10-dioxo-2-sulfo-9,10- dihydro-anthracen-1- ylazo)-benzoic acid | 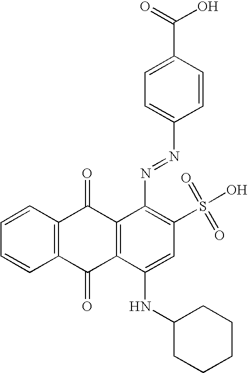 |
| 13 | 2-(4-Cyclohexylamino- 9,10-dioxo-2-sulfo-9,10- dihydro-anthracen-1- ylazo)-benzoic acid | 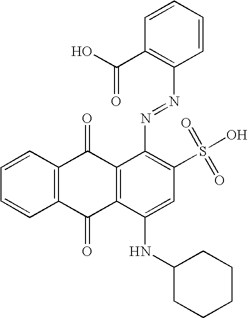 |
| 14 | 4-Cyclohexylamino-1-(5- fluoro-2-methoxy- phenylazo)-9,10-dioxo- 9,10-dihydro-anthracene- 2-sulfonic acid | 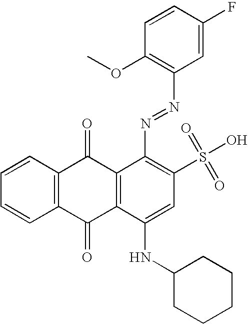 |
| 15 | 3-(4-Cyclohexylamino- 9,10-dioxo-2-sulfo-9,10- dihydro-anthracen-1- ylazo)-4-methoxy- benzoic acid | 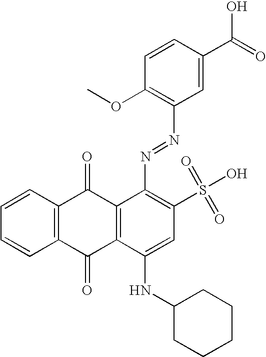 |
| 16 | 3-(4-Cyclohexylamino- 9,10-dioxo-2-sulfo-9,10- dihydro-anthracen-1- ylazo)-4-methoxy- benzoic acid methyl ester | 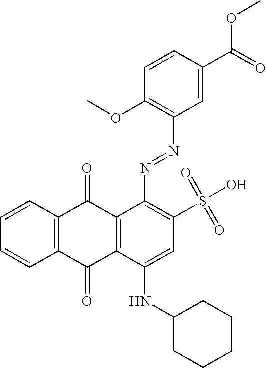 |
| 17 | 4-Cyclohexylamino-9,10- dioxo-1-(2-phenoxy- phenylazo)-9,10-dihydro- anthracene-2-sulfonic acid | 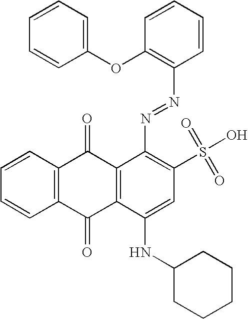 |

### Example 2

### Comparison of Some Characteristics of the Compounds According to the Invention

Table 4 shows some characteristics of compounds in their reaction medium.

TABLE 4

| | | | DO at λₘₐₓ | | |
|---|---|---|---|---|---|
| Dye | Color | λₘₐₓ (nm) | 1* | 2** | 3*** |
| Compound 1 | burgundy | 500 | 0.2 | 8 | 1.73 |
| Compound 2 | violet | 550 | 0.6 | | |
| Compound 3 | fuchsia | 500 | 0.2 | | |
| Compound 4 | fuchsia | 545 | | | 2.75 |
| Compound 6 | fuchsia | 540 | | | 2.35 |
| Compound 8 | fuchsia/burgundy | 470 | | | 3.20 |
| | | 550 | | | 2.85 |
| Compound 9 | violet | 555 | | | 2.58 |
| Compound 10 | violet | 550 | | | 2.73 |
| Compound 11 | fuchsia | 525 | | | 2.15 |
| Compound 12 | fuchsia | 540 | | | 2.36 |
| Compound 13 | violet | 520 | | | 1.83 |
| Compound 14 | fuchsia | 530 | | | 1.97 |
| Compound 15 | fuchsia | 525 | | | 1.95 |
| Compound 16 | mauve | 515 | | | 1.87 |

1* synthesis following protocol N
2** synthesis following the protocol R
3** synthesis following the protocol O

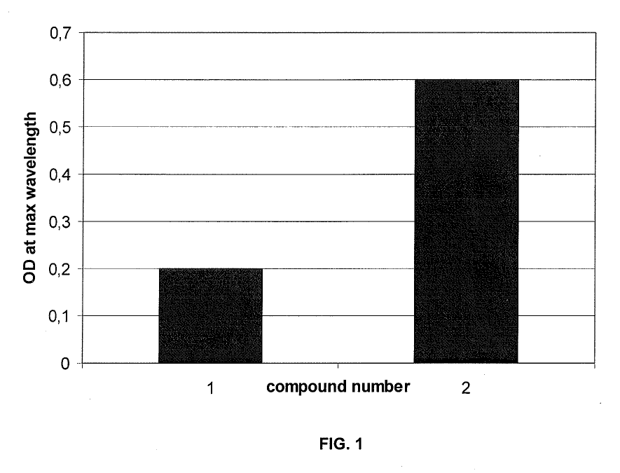

FIG. 1 shows the optical density measured at maximum wavelength for some compounds of the invention (in the reaction medium) prepared according to protocol N.

FIG. 2 shows the optical density measured at maximum wavelength for some compounds of the invention (in the reaction medium) prepared according to protocol O.

### Examples 3

### Results of Dyeing With Compound 1, Compound 2 and Compound 4

Polyamide knitted fabric dyeing—Knitted polyamide 6 was immersed in a dye bath at 40° C., containing the dye solution (0.21%, 0.42%, 2.5% omf), Setacid VS-N (buffering agent—2 g L−1) and Setalan PM-8 (leveling agent—1 g L−1). The liquor ratio was 20/1. The pH was adjusted to 6.5 to 7.5 by adding sodium bicarbonate (0.2-0.3 g L−1). The temperature was raised to 105° C. over 40 min. The dyeing was continued at 105° C. for 40 min. The final pH of the dyebath was between 4.2 and 4.3. The dyed pieces were there rinsed twice at 40 and 25° C. for 5 min (liquor ratio 20/1). Fabrics dyed using 2.5% on mass of fabric (omf) were used for fastness tests as their depth corresponded to the standard depth scale as published by the society of dyers and colorists.

Shade was characterized by reflectance measurements with DataColor SF600 spectrophotometer with 10 nm band width using U.V. excluded and specular included measurements. Spectral range was from 360 to 700 nm. Light source was Pulsed Xenon filtered to appropriated to D65 and optical geometry was dual beam, the angle of viewing was 8° in 6 inches diameter sphere with an aperture size of 26 mm measured and 30 mm illuminated. The Software was Hunter Lab She Lyn.

Polyamide yarn dyeing—Polyamide 6.6 yarn was immersed in an acid dye bath (pH 5.2) containing 1% omf leveling agent Alvolan UL75 at 40° C., liquor ratio 10/1. Compound 1 was added and temperature was raised at 0.5° C. min⁻¹ to 100° C. during 60 min. The bath was then cooled (3° C. min⁻¹) to 60° C. The dyed pieces were there rinsed twice at 40° C. and 25° C. for 5 min (liquor ratio 10/1).

Leather dyeing—Bovine wet blue samples of approximately 30 g were neutralized to pH about 7.4 into a small glass drum using sodium formate (2% omf) and sodium bicarbonate (2.5% omf), liquor ratio 2/1, at 35° C. for 100 min. After the neutralization step, the drum was drained and the samples were retanned using mimosa (3% omf), liquor ratio 2/1 at 35° C. for 30 min. The dyeing was carried out with compound 1 (3% omf) during 30 min. The dye uptake of the leather was determined using a UV-Visible Spectrophotometer (Varian Cary 50 Bio). The samples were then fat liquored using Ombella WR (5% omf) during 40 min at 50° C. and fixed using formic acid (0.2% omf) during 30 min at 50° C.

The fastness properties of the dyeings (textile and leather) were measured in accordance with ISO standards mentioned in table 5. The results are shown in Table 5 based on visual assessment described in ISO105-A02: 1993 textiles—test for colorfastness—part A02. On this scale, a rating of 5 means no color change and a rating of 1 means a high color change.

TABLE 5
Dyeing properties on polyamide 6 knitted fabric

| | | |
|---|---|---|
| Standard depth: | 1/1 | (2.4% omf) |
| Light fastness | 3 | ISO 105 B02 |
| Washing fastness | | ISO 105 C06: A2S |
| Color change | 4/5 | |

| | | | | | | |
|---|---|---|---|---|---|---|
| | Cellulose | | | | | |
| Staining | acetate | Cotton | Nylon | Polyester | Acrylic | Wool |
| | 4/5 | 3 | 3 | 4/5 | 4/5 | 4 |

| | | |
|---|---|---|
| Alkaline perspiration fastness | | ISO 105 E04 |
| Color change | 3/4 | |

| | | | | | | |
|---|---|---|---|---|---|---|
| | Cellulose | | | | | |
| Staining | acetate | Cotton | Nylon | Polyester | Acrylic | Wool |
| | 2/3 | 2 | 2 | 3 | 3 | 3 |

Dyeing properties on polyamide 6.6 yarn

| | | | |
|---|---|---|---|
| (1% omf) | | | |
| Light fastness | | 2 | ISO 105 B02 |
| Water fastness | | 5 | ISO 105 E01 |
| Perspiration fastness | Acid | 4/5 | ISO 105 E04 |
| | alkaline | 4/5 | ISO 105 E04 |
| Washing fastness | 40° C. | 5 | ISO 105 C01 |
| | 60° C. | 5 | ISO 105 C03 |
| | 60° C. | 4/5 | ISO 105 C06 C1S |
| Chlorine fastness | | 4/5 | ISO 105 E03 100 mg L⁻¹ |
| Crocking | Dry | 4/5 | ISO 105 X12 |
| | Wet | 4/5 | ISO 105 X12 |

Dyeing properties on bovine wet blue leather

| | | |
|---|---|---|
| (3% omf) | | |
| Light fastness | 3 | BS EN ISO 11641 |
| Fastness to water spotting | 5 | BS EN ISO 11641 |
| PVC migration staining | 3/4 | BS EN ISO 11641 |
| Water fastness | | BS EN ISO 11641 |

| | | | | | | |
|---|---|---|---|---|---|---|
| | Cellulose | | | | | |
| Staining | acetate | Cotton | Nylon | Polyester | Acrylic | Wool |
| Grain | 4 | 3/4 | 1/2 | 4 | 4/5 | 2/3 |
| Flesh | 3 | 2-3 | 1 | 4/5 | 4/5 | 2 |

| | |
|---|---|
| Perspiration fastness | BS EN ISO 11641 |

| | | | | | | |
|---|---|---|---|---|---|---|
| | Cellulose | | | | | |
| Staining | acetate | Cotton | Nylon | Polyester | Acrylic | Wool |
| Grain | 3 | 3 | 1 | 4 | 4/5 | 2 |
| Flesh | 2 | 2/3 | 1 | 3/4 | 3/4 | 2 |

The dyeing properties of compounds 1, 2 and 4 were tested on samples of knitted fabric.

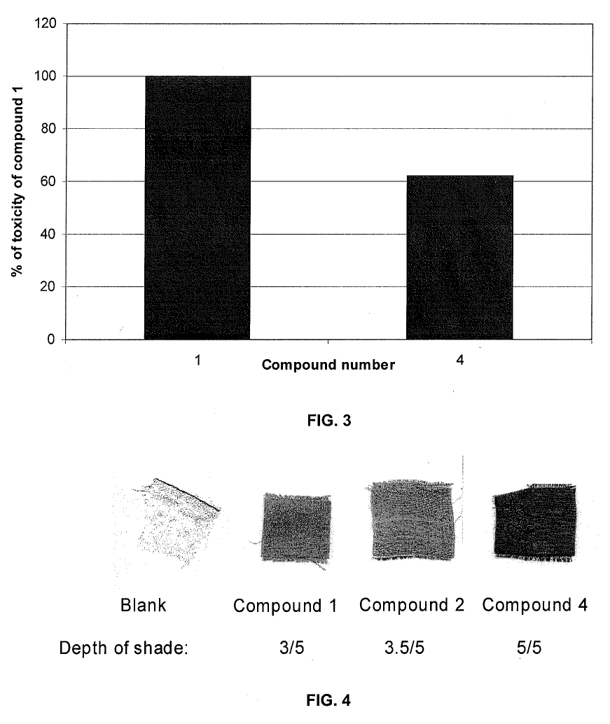

Knitted silk was immersed in a dye bath at 40° C., containing the dye solution (2% omf) buffered at pH 6.5 with acetic acid. The liquor ratio was 20/1. The dyeing was continued at 95° C. for 1 hour. Salt was added (100% omf) and the dyeing was continued at 95° C. for 1 hour. The dyed pieces were rinsed 3 times at 40° C. with water. After drying, color of the sample was evaluated visually. The results are shown in FIG. 4.

FIG. 4 shows pictures of silk samples dyed with compounds 1, 2 and 4. The white silk fabric was dyed in pink by the compound 1, in bluish grey by the compound 2 and in bright red/purple by the compound 4. Depth of shade was evaluated as medium for compounds 1 (3/5) and 2 (3.5/5) and as strong for compound 4 (5/5).

### Example 4

### Toxicity and Mutagenicity Properties of a Compound According to the Present Invention, and Comparison With the Characteristics of Azo Dyes Available on the Market

The toxicity of compound 1 was compared with the toxicity of disperse red 1, reactive red 4, direct red 28, and acid red 299, which are products available on the market.

Toxicity was assessed on human intestinal cells (Caco-2), bacterial cells (Lumistox assays) and fish eggs cells. Mutagenicity was assessed through classical Ames test. The results are shown in Table 6.

TABLE 6

| | | Disperse | Reactive | Direct Red | Acid Red |
|---|---|---|---|---|---|
| | Compound 1 | Red 1 | Red 4 | 28 | 299 |
| CAS-No | | 2872-52-8 | 17681-50-4 | 573-58-0 | 57741-47-6 |
| Caco-2 cytotoxicity test | >1 | >1 | 0.7 ± 0.06 | 0.3 ± 0.02 | >1 |
| (IC₅₀ in g L⁻¹) | | | | | |
| Ames test: mutagenicity | Negative | Negative | Negative | Positive* | Positive |
| Fish egg test | 294 | 250-500 | 500-1000 | <30 | 250-500 |
| (IC₅₀ in mg L⁻¹) | | | | | |
| Lumistox test | 41.6** | 61.3*** | 25.9 | 3.4 | 50.2 |
| % of luminescence | | | | | |
| inhibition at maximal OD | | | | | |

*Published data.
**up to a maximal optical density (OD) of 1800.
***saturated solution

The results showed that the compounds presented a low toxicity and no mutagenicity in the range tested.

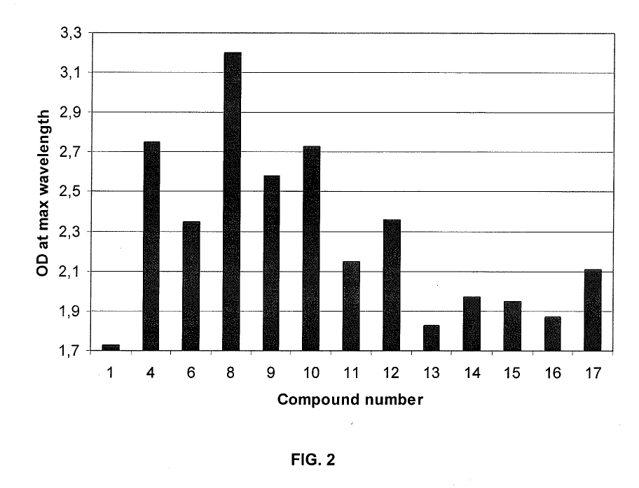

The toxicity of compound 4 has been measured on Caco-2 human cells as previously described, and compared with the toxicity of compound 1. The results are shown in FIG. 3. It can be seen that the toxicity of compound 4 is 30% less that the toxicity of the compound 1, which itself was shown to present a low toxicity with regards to other molecules available on the market.

What is claimed is:

1. A compound having the structural Formula (I) or (II), a tautomer, a quaternary form, or a salt thereof, 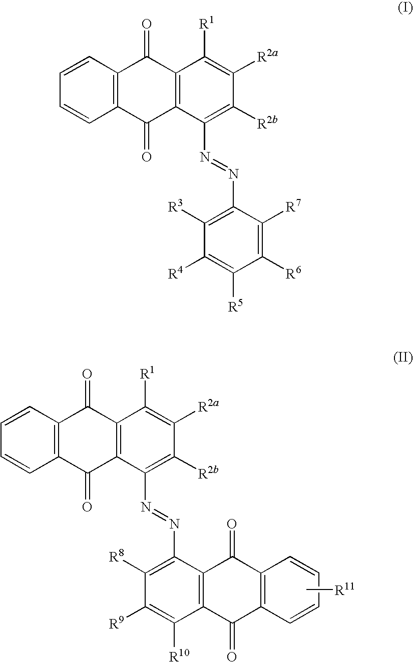
wherein
R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂;
R^{2a} is hydrogen or —SO₃H;
R^{2b} is hydrogen or —SO₃H;
wherein at least one of R^{2a} or R^{2b} is —SO₃H;
R³ is selected from the group consisting of hydrogen, halogen, —SO₃H, C₁₋₆alkyl, amino, C₁₋₆alkylamino, C₆₋₁₂arylamino, C₁₋₆alkoxy, C₆₋₁₀aryloxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂;
R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, and C₁₋₆alkylaminocarbonyl;
R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁶ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, —C(OH)═CH—OH, and —NH-phenylene-SO₂—CH═CH₂;
R⁸ is hydrogen or —SO₃H;
R⁹ is hydrogen or —SO₃H;
R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and
R¹¹ is selected from the group consisting of hydrogen, C₁₋₆alkyl, amino, halogen, hydroxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl.

2. A compound according to claim 1,
wherein
R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂;
R^{2a} is hydrogen or —SO₃H;
R^{2b} is hydrogen or —SO₃H;
wherein at least one of R^{2a} or R^{2b} is —SO₃H;
R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂;
R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁶ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, —C(OH)═CH—OH, and —NH-phenylene-SO₂—CH═CH₂;
R⁸ is hydrogen or —SO₃H;
R⁹ is hydrogen or —SO₃H;
R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and
R¹¹ is selected from the group consisting of hydrogen, C₁₋₆alkyl, amino, halogen, hydroxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl.

3. The compound according to claim 1 wherein at least one of R⁸ and R⁹ is —SO₃H.

4. The compound according to claim 1, wherein R^{2a} is —SO₃H; and R^{2b} is hydrogen.

5. The compound according to claim 1, wherein R⁸ is —SO₃H; and R⁹ is hydrogen.

6. The compound according to claim 1, wherein R¹⁰ is selected from the group consisting of C₃₋₈cycloalkylamino, amino, C₁₋₆alkylamino, C₆₋₁₂arylamino, haloC₆₋₁₂arylamino, and —N═N—C₆₋₁₂arylene-SO₃H.

7. The compound according to claim 1, wherein R¹¹ is hydrogen.

8. The compound according to claim 1, wherein R¹ is selected from the group consisting of hydrogen, C₁₋₆alkyl, C₃₋₈cycloalkylamino, amino, hydroxyl, C₁₋₆alkylamino, C₆₋₁₂arylamino, haloC₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, —N═N—CH═CH—C₆₋₁₂arylene-hydroxyl, —N═N—C₆₋₁₂arylene-SO₃H, and —SO₂—CH═CH₂;
R^{2a} is hydrogen or —SO₃H;
R^{2b} is hydrogen or —SO₃H;
wherein at least one of R^{2a} or R^{2b} is —SO₃H;
R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, carboxyl, aminocarbonyl, C₁₋₆alkylaminocarbonyl, and —NH-phenylene-SO₂—CH═CH₂;
R⁴ is selected from the group consisting of hydrogen, halogen, amino, —SO₃H, C₁₋₆alkyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁵ is selected from the group consisting of hydrogen, halogen, amino, nitro, —SO₃H, C₁₋₆alkyl, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁶ is selected from the group consisting of hydrogen, halogen, amino, and C₁₋₆alkyl;
R⁷ is selected from the group consisting of hydrogen, halogen, carboxyl, —C(OH)═CH—OH, amino, and C₁₋₆alkyl;
R⁸ is hydrogen or —SO₃H;
R⁹ is hydrogen or —SO₃H; wherein at least one of R⁸ or R⁹ is —SO₃H;
R¹⁰ is selected from the group consisting of hydrogen, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and
R¹¹ is hydrogen.

9. The compound according to claim 1, wherein R⁶ is hydrogen.

10. The compound according to claim 1, wherein R¹ is selected from the group consisting of hydrogen, C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl, and —N═N—C₆₋₁₂arylene-SO₃H.

11. The compound according to claim 1, wherein
R¹ is selected from the group consisting of hydrogen, C₃₋₈cycloalkylamino, amino, C₁₋₆alkylamino, C₆₋₁₂arylamino, haloC₆₋₁₂arylamino, —N═N—CH═CH—C₆₋₁₂arylene-hydroxyl, and —N═N—C₆₋₁₂arylene-SO₃H;
R^{2a} is —SO₃H; and R^{2b} is hydrogen;
R³ is selected from the group consisting of hydrogen, halogen, —SO₃H, C₁₋₆alkyl, amino, C₁₋₆alkylamino, C₆₋₁₂arylamino, C₁₋₆alkoxy, C₁₋₆alkoxyC₁₋₆alkyl, C₁₋₆alkoxycarbonyl, and carboxyl;
R⁴ is selected from the group consisting of hydrogen, —SO₃H, C₁₋₆alkyl, amino, halogen, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁵ is selected from the group consisting of hydrogen, halogen, —SO₃H, C₁₋₆alkyl, amino, nitro, hydroxyl, carboxyl, C₁₋₆alkylamino, C₆₋₁₂arylamino, C₁₋₆alkoxy, C₁₋₆alkoxyC₁₋₆alkyl, aminocarbonyl, and C₁₋₆alkylaminocarbonyl;
R⁶ is hydrogen;
R⁷ is hydrogen, carboxyl or —C(OH)═CH—OH;
R⁸ is hydrogen or —SO₃H;
R⁹ is hydrogen or —SO₃H;
R¹⁰ is selected from the group consisting of hydrogen, halogen, C₁₋₆alkyl, amino, hydroxyl, C₁₋₆alkylamino, C₃₋₈cycloalkylamino, C₆₋₁₂arylamino, C₁₋₆alkoxy, C₁₋₆alkoxyC₁₋₆alkyl, aminocarbonyl, and C₁₋₆alkylaminocarbonyl; and
R¹¹ is hydrogen.

12. A process for the production of a compound according to claim 1, comprising the step of: coupling an amine of formula (Ia) with a compound of formula (Ib) or (IIb) in the presence of an oxidoreductase enzyme; 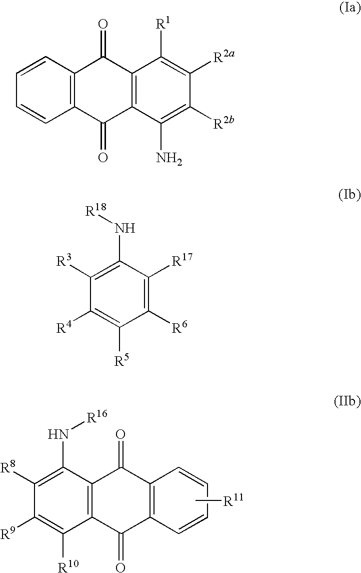
wherein R¹⁶ is selected from the group comprising hydrogen, C₁₋₆cycloalkyl, C₆₋₁₀aryl, haloC₆₋₁₀aryl, 9,10-dioxoanthracenyl, or 4-p-tolyloxysulfonyloxy-phenyl;
R¹⁷ is selected from the group consisting of hydrogen, halogen, amino, C₁₋₆alkyl, C₁₋₆alkylamino, C₆₋₁₂arylamino, C₁₋₆alkoxy, C₁₋₆alkoxyC₁₋₆alkyl, C₁₋₆alkoxycarbonyl, aminocarbonyl, C₁₋₆alkylaminocarbonyl, carboxyl and —NH-phenylene-SO₂—CH═CH₂; and
R¹⁸ is selected from hydrogen, or C₆₋₁₂aryl; or
NH—R¹⁸ and R¹⁷ together with the carbon atom to which they are attached form a 5 membered heteroaryl or heterocyclyl ring, each ring being optionally substituted with one or two substituents selected from C₁₋₆alkylcarbonyloxy or carboxyl;
wherein R¹, R^{2a}, R^{2b}, R³, R⁴, R⁵, R⁶, R⁸, R⁹, R¹⁰, and R¹¹ have the same meaning as defined in claim 1.

13. The process according to claim 12, wherein the oxido reductase enzyme is selected from the group consisting of laccase, peroxidase, cellobiose dehydrogenase, and tyrosinase.

14. The process according to claim 13, wherein the laccase is produced by white rot fungi.

15. The process according to claim 12, wherein the enzyme is a free enzyme or an immobilized enzyme.

16. The process according to claim 15, wherein said enzyme is immobilized on a solid support.

17. The process according to claim 16, wherein said solid support is selected from glass beads, perlite, montmorillonite, alginate, carrageenan.

18. The process according to claim 12, further comprising the step of at least sequentially agitating or aerating, or oxygenating the reaction mixture or performing the reaction under overpressure of oxygen.

19. A method of dyeing an article comprising the step of contacting said article with a compound according to claim 1.

20. The method according to claim 19, wherein said article is made of a material selected from a fabric, yarn, fiber, garment or film made of a material selected from the group consisting of fur, hair, hide, leather, silk, wool, cationic polysaccharide, and synthetic polyamide.
